(19) United States
(12) Patent Application Publication
(10) Pub. No.: US 2025/0364100 A1
(43) Pub. Date: Nov. 27, 2025

(54) SYSTEMS AND METHODS OF EEG-BASED BIOMARKER DISCOVERY AND COMMERCIALIZATION FOR PERSONALIZED DIAGNOSIS AND TREATMENT OF BRAIN DISORDERS

(71) Applicant: Neuroscience Software Inc. DBA Brainify.AI, New York City, NY (US)

(72) Inventors: Mariam Khayretdinova, New York City, NY (US); Ivan Mishanin, New York, NY (US); Andrey Kiryasov, New York City, NY (US); Polina Pshonkovskaya, New York City, NY (US); Maxim Popov, New York City, NY (US); Andrey Zhdanov, Helsinki (FI); Aleksei Matveev, New York City, NY (US)

(73) Assignee: Neuroscience Software Inc. DBA Brainify.AI, New York City, NY (US)

(21) Appl. No.: 19/216,386

(22) Filed: May 22, 2025

Related U.S. Application Data
(60) Provisional application No. 63/650,747, filed on May 22, 2024.

(51) Int. Cl. G16H 20/10; A61B 5/00; A61B 5/372; G16H 10/20; G16H 10/60; G16H 15/00; G16H 50/20; G16H 50/70

(52) U.S. Cl. CPC G16H 20/10; A61B 5/372; A61B 5/7203; A61B 5/7267; G16H 10/20; G16H 10/60; G16H 15/00; G16H 50/20; G16H 50/70

(57) ABSTRACT

The present disclosure provides a method of EEG-based biomarker discovery and commercialization for personalized diagnosis and treatment of brain disorders. Further, the method may include receiving, using a communication device, a brain-wave data from a diagnostic device. Further, the brain-wave data corresponds to a graphical representation of an electrical activity of a brain of an individual. Further, the individual may be receiving at least one therapeutic for at least one brain disorder. Further, the method may include analyzing, using a processing device, the brain-wave data based on an artificial intelligence (AI) model. Further, the method may include generating, using the processing device, at least one output data based on the analyzing. Further, the method may include transmitting, using the communication device, the at least one output data to at least one device.

### FIELD OF DISCLOSURE

The present disclosure generally relates to a field of data processing. More specifically, the present disclosure related to systems and methods of EEG-based biomarker discovery and commercialization for personalized diagnosis and treatment of brain disorders.

### BACKGROUND

Mental, psychiatric, neurological, and neurodegenerative disorders represent a significant global health burden affecting billions of individuals worldwide. Neurological disorders alone affect 3.4 billion people (43% of the world population), cause 11.1 million deaths annually, and are responsible for 443 million disability-adjusted life years (DALYs), making them the leading cause of global disease burden. Major contributors include stroke, dementia, and migraine. Mental disorders affect nearly one in four people worldwide, with intervention rates below 25% globally. According to the World Health Organization (WHO), approximately 450 million people are affected by mental health conditions globally, with over 300 million suffering from depression. Alarmingly, 60% of mentally-ill adults receive no mental health services. Neurodegenerative diseases show concerning trends, with dementia affecting 55 million people worldwide and one new case occurring every 3 seconds. Seventy-five percent of dementia cases go undiagnosed globally, with annual costs of $1.3 trillion projected to reach $2.8 trillion by 2030. Parkinson's disease prevalence increased by 155.51% from 1990-2019, with mortality rates rising from 5.3 to 9.8 per 100,000 between 1999-2020.

Traditional treatments for these disorders, such as pharmacological interventions and psychotherapy, face significant challenges. About one-third of patients with depression don't respond adequately to available treatments. Current diagnostic and treatment approaches impose substantial burdens on patients and clinicians, with limited new drug development in recent years. There is a critical need for personalized approaches using objective measurements rather than subjective assessments, as one-size-fits-all treatments show variable effectiveness.

Pharmaceutical companies face numerous challenges in developing effective treatments. Despite increased R&D spending, fewer new psychiatric drugs receive approval compared to other therapeutic areas. Reduced assay sensitivity in clinical outcome measures has contributed to the exodus of pharmaceutical companies from central nervous system (CNS) drug development. Industry influence has resulted in overestimation of medication effectiveness and underreporting of side effects, with many companies prioritizing marketing over R&D-7 of 10 top pharmaceutical companies spend more on sales than research.

Clinical trials for these disorders show discouragingly low success rates. Oncology trials have 3.4% success rates (compared to 5.1% in previous studies), while Alzheimer's has a staggering 99.6% clinical trial failure rate. Major depressive disorder trials show only 50% probability of statistical significance versus placebo (compared to expected 80-90% from statistical powering). Despite over $100 billion invested in R&D for psychiatric treatment over the past 10 years, only 18 drugs have received FDA approval.\

There is a strong unmet need for improved systems and methods of facilitating therapy across mental, psychiatric, neurological, and neurodegenerative disorders that can provide more personalized, efficient approaches to diagnosis and treatment.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of EEG-based biomarker discovery and commercialization for personalized diagnosis and treatment of brain disorders. Further, the method may include receiving, using a communication device, a brain-wave data from a diagnostic device. Further, the diagnostic device may include an electroencephalogram (EEG) capturing device. Further, the brain-wave data corresponds to a graphical representation of an electrical activity of a brain of an individual. Further, the individual may include a patient and a subject. Further, the brain-wave data may include an electroencephalogram (EEG) reading of the individual. Further, the individual may be in a resting state. Further, the individual may be undergoing an Event-Related Potential (ERP) session. Further, the individual may be receiving at least one therapeutic for at least one brain disorder. Further, the at least one therapeutic may include a mental therapy, a neurological therapy, a neurodegenerative therapy, and at least one drug. Further, the at least one brain disorder may include at least one of a mental condition, a psychiatric condition, a neurological condition, and a neurodegenerative condition. Further, the method may include analyzing, using a processing device, the brain-wave data based on an artificial intelligence (AI) model. Further, the brain-wave data may be analyzed to identify one or more electroencephalogram (EEG) biomarkers. Further, the one or more EEG biomarkers may be indicative of a condition of the individual. Further, the one or more EEG biomarkers may facilitate monitoring of a condition of the individual receiving the at least one therapeutic. Further, the AI model may be trained on a brain-wave dataset associated with a mental disorder, a psychiatric disorder, a neurological disorder, and a neurodegenerative disorder, and at least one additional data comprising a clinical data, a demographic data, an EMR (electronic medical record)/EHR (electronic health record) data, of the individual. Further, the method may include generating, using the processing device, at least one output data based on the analyzing. Further, the at least one output data may be indicative a response of the individual to the at least one therapeutic, and an influence of the at least one therapeutic on the individual. Further, the method may include transmitting, using the communication device, the at least one output data to at least one device.

The present disclosure provides a system of EEG-based biomarker discovery and commercialization for personalized diagnosis and treatment of brain disorders. Further, the system may include a communication device. Further, the communication device may be configured for receiving a brain-wave data from a diagnostic device. Further, the diagnostic device may include an electroencephalogram (EEG) capturing device. Further, the brain-wave data corresponds to a graphical representation of an electrical activity of a brain of an individual. Further, the individual may include a patient and a subject. Further, the brain-wave data may include an electroencephalogram (EEG) reading of the individual. Further, the individual may be in a resting state. Further, the individual may be undergoing an Event-Related Potential (ERP) session. Further, the individual may be receiving at least one therapeutic for at least one brain disorder. Further, the at least one therapeutic may include a mental therapy, a neurological therapy, a neurodegenerative therapy, and at least one drug. Further, the at least one brain disorder may include at least one of a mental condition, a psychiatric condition, a neurological condition, and a neurodegenerative condition. Further, the communication device may be configured for transmitting at least one output data to at least one device. Further, the system may include a processing device communicatively coupled with the communication device. Further, the processing device may be configured for analyzing the brain-wave data based on an artificial intelligence (AI) model. Further, the brain-wave data may be analyzed to identify one or more electroencephalogram (EEG) biomarkers. Further, the one or more EEG biomarkers may be indicative of a condition of the individual. Further, the one or more EEG biomarkers may facilitate monitoring of a condition of the individual receiving the at least one therapeutic. Further, the AI model may be trained on a brain-wave dataset associated with a mental disorder, a psychiatric disorder, a neurological disorder, and a neurodegenerative disorder, and at least one additional data comprising a clinical data, a demographic data, an EMR (electronic medical record)/EHR (electronic health record) data, of the individual. Further, the processing device may be configured for generating the at least one output data based on the analyzing. Further, the at least one output data may be indicative a response of the individual to the at least one therapeutic, and an influence of the at least one therapeutic on the individual.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 illustrates a flowchart of a method 300 of EEG-based biomarker discovery and commercialization for personalized diagnosis and treatment of brain disorders, in accordance with some embodiments.

FIG. 4 illustrates a flowchart of a method 400 of EEG-based biomarker discovery and commercialization for personalized diagnosis and treatment of brain disorders including analyzing, using the processing device 704, the pre-processed brain-wave data, in accordance with some embodiments.

FIG. 5 illustrates a flowchart of a method 500 of EEG-based biomarker discovery and commercialization for personalized diagnosis and treatment of brain disorders including generating, using the processing device 704, a report data, in accordance with some embodiments.

FIG. 6 illustrates a flowchart of a method 600 of EEG-based biomarker discovery and commercialization for personalized diagnosis and treatment of brain disorders including determining two or more targeted individuals, in accordance with some embodiments.

FIG. 7 illustrates a block diagram of a system 700 of EEG-based biomarker discovery and commercialization for personalized diagnosis and treatment of brain disorders, in accordance with some embodiments.

FIG. 8 illustrates a block diagram of the system 700, in accordance with some embodiments.

FIG. 9 illustrates a flowchart of a method 900 of EEG-based biomarker discovery and commercialization for personalized diagnosis and treatment of brain disorders including modifying each of the plurality of brain-wave data, in accordance with some embodiments.

FIG. 10 illustrates a flowchart of a method 1000 of EEG-based biomarker discovery and commercialization for personalized diagnosis and treatment of brain disorders including modifying each of the plurality of modified brain-wave data, in accordance with some embodiments.

FIG. 11 illustrates a flowchart of a method 1100 of EEG-based biomarker discovery and commercialization for personalized diagnosis and treatment of brain disorders including filtering the artifact noise data from the brain-wave data to obtain a filtered brain-wave data, in accordance with some embodiments.

FIG. 12 illustrates a flowchart of a method 1200 of EEG-based biomarker discovery and commercialization for personalized diagnosis and treatment of brain disorders including generating the pre-processed brain-wave data, in accordance with some embodiments.

FIG. 13 illustrates a flowchart of a method 1300 of EEG-based biomarker discovery and commercialization for personalized diagnosis and treatment of brain disorders including modifying each of a plurality of pre-processed brain-wave data, in accordance with some embodiments.

FIG. 14 illustrates a flowchart of a method 1400 of EEG-based biomarker discovery and commercialization for personalized diagnosis and treatment of brain disorders including analyzing, using the processing device 704, the questionnaire data, in accordance with some embodiments.

FIG. 15 illustrates a flowchart of a method 1500 of EEG-based biomarker discovery and commercialization for personalized diagnosis and treatment of brain disorders including generating, using the processing device 704, a treatment data, in accordance with some embodiments.

FIG. 16 illustrates a flowchart of a method 1600 of EEG-based biomarker discovery and commercialization for personalized diagnosis and treatment of brain disorders including analyzing, using the processing device 704, the baseline brain-wave data, in accordance with some embodiments.

FIG. 17 illustrates a flowchart of a method 1700 of EEG-based biomarker discovery and commercialization for personalized diagnosis and treatment of brain disorders including analyzing, using the processing device 704, the baseline-questionnaire data, in accordance with some embodiments.

FIG. 18 illustrates a system 1800 for identifying EEG-based pharmacologically significant biomarker, in accordance with some embodiments.

FIG. 19 illustrates a system 1900 for predicting effectiveness of a drug trial on participants, in accordance with some embodiments.

FIG. 20 illustrates a system and method pathway 2000 for phase II trials, in accordance with some embodiments.

FIG. 21 illustrates a system and method pathway 2100 for phase III trials and FDA regulation, in accordance with an exemplary embodiment.

FIG. 22 illustrates an equipment and tools 2200 utilized during data collection by the system and method, in accordance with an exemplary embodiment.

FIG. 23 illustrates a technical architecture 2300 of the system and method, in accordance with some embodiments.

FIG. 24 illustrates a flowchart of a method 2400 of EEG-based biomarker discovery and commercialization for personalized diagnosis and treatment of brain disorders including generating, using the processing device 704, a brain-wave dataset using a data-whitening model, in accordance with some embodiments.

FIG. 25 illustrates a flowchart of a method 2500 of EEG-based biomarker discovery and commercialization for personalized diagnosis and treatment of brain disorders including clustering the plurality of brain-wave data in a multi-dimensional space, in accordance with some embodiments.

FIG. 26 illustrates a flowchart of a method 2600 of EEG-based biomarker discovery and commercialization for personalized diagnosis and treatment of brain disorders including generating, using the processing device 704, a score data, in accordance with some embodiments.

FIG. 27 illustrates a convolutional neural network 2700, according to an exemplary embodiment.

FIG. 28 illustrates convolutional neural network layers 2800, in accordance with an exemplary embodiment.

FIG. 29 illustrates a VGG net 2900, in accordance with some embodiments.

FIG. 30 illustrates a convolution layer filtering 3000, in accordance with some embodiments.

FIG. 31 illustrates a pooling layer function 3100, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein-as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes Brianify.AI. Further, the present disclosure describes methods and systems of the Brianify.AI. Further, the Brianify.AI is an AI/ML electroencephalography (EEG) biomarker platform for depression treatment response prediction that increases the likelihood of new drug approval by 80% and reduces R&D costs. Further, the methods and systems are designed to enhance the efficiency of clinical trials and improve patient outcomes. Further, the methods and systems identify biomarkers that predict treatment response during clinical phase 2, use them to select patients for clinical phase 3 trials, and predict placebo response in phase 2 and phase 3 to increase the likelihood of drug approval.

The development of biomarkers capable of predicting treatment responses is a promising area of research. These biomarkers can provide insights into how an individual's biological characteristics influence their reaction to specific therapies. Electroencephalography (EEG), a non-invasive method for recording brain activity, offers unique insights into neural states and cognitive processes across multiple disorders: psychiatric conditions (bipolar disorder, schizophrenia), neurological disorders (Parkinson's, epilepsy), and neurodegenerative diseases (Alzheimer's, mild cognitive impairment). Key advantages include non-invasiveness, cost-effectiveness, and high temporal resolution, allowing for real-time diagnosis, treatment response monitoring, and disease progression tracking through various measures like spectral analysis, entropy, and functional connectivity.

Brainify.AI is an innovative, EEG-based AI/ML biomarker platform designed to revolutionize the development of novel treatments for depression. By harnessing the power of AI and ML, Brainify.AI increases the likelihood of approval by 80% and reduces R&D costs, ultimately accelerating the process of bringing effective treatments to market. The platform comprises three interconnected products—PlaceboInsight AI, TherapyInsight AI, and CleanSpectrum AI-that work in tandem to streamline clinical trials and optimize patient selection and stratification, based on their predicted response to novel therapeutics and placebo. The following embodiments define the scope of each product.

### PlaceboInsight AI: Revolutionizing Clinical Trial Efficiency

PlaceboInsight AI optimizes clinical trial success rates by identifying potential placebo responders or by predicting subjects' prognostic scores (expected score if the subject were to receive placebo) using advanced AI/ML models. This targeted approach simplifies trial design, reduces costs, and accelerates the drug development process. PlaceboInsight Al's transfer learning techniques enable rapid adaptation to a wide range of diseases, making it a versatile solution for clinical research across multiple therapeutic areas.

### TherapyInsight AI: Personalized Treatment Prediction

TherapyInsight AI identifies subjects most likely to respond to new therapeutics during Phase III clinical trials as well as in a clinical setting, leveraging transfer learning technology and a range of AI/ML models. By predicting treatment response and identifying suitable candidates for Phase III trials, TherapyInsight AI streamlines the clinical trial process, increases the likelihood of positive trial outcomes, and accelerates the development of effective treatments for psychiatric disorders. In a clinical setting, TherapyInsight AI will serve as a companion diagnostic test to predict a patient's response to a particular treatment, thus assisting healthcare professionals in selecting the most suitable treatment option. Its adaptability makes it a versatile solution for clinical research across multiple therapeutic areas.

### CleanSpectrum AI: Enhancing EEG Data Quality for Effective AI/ML Applications

CleanSpectrum AI addresses the challenges of using EEG data in AI/ML models by harmonizing datasets from different sources, preventing data leakage, and preserving clinically relevant signals. CleanSpectrum AI might include Data Whitening model. Its cutting-edge AI/ML techniques enable researchers to obtain meaningful insights from their AI/ML models, leading to improved diagnostic assessments and treatment predictions for various neurological and psychiatric disorders. Together, these three products create a comprehensive, interconnected platform that transforms clinical trials and accelerates the development of novel treatments for depression. By combining advanced AI and ML technologies with innovative approaches to data analysis and prediction, Brainify.AI is posed to revolutionize the drug development process, paving the way for more effective treatments and improved patient outcomes in the field of psychiatric disorders.

Pharmaceutical companies face challenges such as patent exclusivity loss, declining revenues, and the complex nature of depression, which contribute to high placebo effects in clinical trials. The industry grapples with increasing R&D costs, declining productivity, and inefficiencies in clinical treatment and diagnosis. The need for biomarkers in psychiatry is crucial to improve diagnosis and treatment response prediction. Currently, the alternatives for depression diagnosis and treatment rely on subjective questionnaires, underscoring the demand for more accurate and objective solutions.

Mental disorders affect almost 1 billion people worldwide and cost the world economy $2-5 trillion annually, with the US alone spending approximately $220 billion on mental health services.

Pharmaceutical companies face the looming challenge of patent exclusivity loss, which is projected to cause a staggering $110 billion loss in global sales between 2023 and 2030. To counteract this loss, companies must innovate and acquire fresh revenue streams by developing new, effective drugs and treatments. This urgency to innovate highlights the need for solutions that can expedite drug development and approval processes, ensuring a continuous pipeline of novel therapeutics.

The complex and heterogeneous nature of depression, coupled with the high placebo effect in clinical trials, presents significant challenges in developing effective treatments. Pharma companies spend tens of billions of dollars per year on research and development, with at least $10 billion dedicated to Phase 2 and 3 clinical trials annually. However, declining research and development productivity over the past two decades has led to a smaller number of successful drugs. The emergence of specialized diseases and disorders necessitates more costly and time-consuming research, further exacerbating the issue. A solution that can accurately predict treatment response and improve clinical trial outcomes is critical to addressing these challenges and accelerating the development of effective treatments.

The pharmaceutical industry is under growing pressure from a range of environmental issues, including major losses of revenue owing to patent expirations, increasingly cost-constrained healthcare systems, and more demanding regulatory requirements (Paul et al., 2010). A possible solution to overcoming the existing issues in the healthcare and pharmaceutical sectors is improving the quality and quantity of the emerging drugs while also reducing R&D costs. However, the latest reports from the field show a trend in the opposite direction. It would be logical to assume that, after the past half-century of technological and scientific developments, the R&D process would increase the efficacy of emerging medicines. The reality, however, is that the number of approved drugs on the market reduces by half every nine years (Scannell et al., 2012).

Clinics and hospitals face difficulties in effectively treating different cases of depression due to the inability to predict an individual's treatment response. The process of choosing an effective drug can take months, costing healthcare systems additional money and putting patients at high risk. Low accuracy in diagnosing depression (approximately 50%) and low treatment efficacy (less than 33%) further compound the challenges of addressing depression and other mental health disorders. A solution that enhances diagnostic accuracy, predicts treatment response, and improves overall treatment efficacy is crucial to overcoming these obstacles and providing better care to patients suffering from depression.

The field of psychiatry significantly lags behind other fields of medicine with respect to patient diagnosis. While other health-related ailments can be diagnosed via diagnostic imaging or tissue biopsy, psychiatric conditions (i.e., depression) are diagnosed via self-reported symptoms experienced by the patient. The lack of objective diagnostic biomarkers has significantly hindered the development of new therapeutics in the field and has negatively affected the lives of millions of people suffering from mental health conditions. Traditionally, individual differences among patients have been a major obstacle in the development of both psychiatric biomarkers and therapeutics, and heterogeneity among patient populations is a major contributing factor to variability in therapeutic response. The use of segmented biomarkers to selectively identify patients who are more likely to respond to a given therapeutic represents a major paradigm shift towards the advent of personalized medicine.

Phase 2 clinical trials currently suffer from the lowest transition rate among all phases of the drug approval process (28.8%). Excitingly, the implementation of preselection biomarkers has been shown to have the strongest benefit at the Phase 2 stage, with an improvement in the transition success rate to 46.3%.

Additional increases in success rate (from 25.0% to 68.2%) were also seen in Phase 3 trials that implemented the use of preselection biomarkers. Success at the NDA/BLA transition is largely contingent on Phase 3 trial design, so this benefit carries over from that phase.

The Brainify addresses two critical areas within the field of psychiatric drug development by providing objective biomarkers that will facilitate both patient diagnosis and patient preselection for clinical trials.

Currently, there are no biomarkers available to predict treatment response in depression, and clinicians often resort to using treatments with fewer side effects and lower costs.

Depression diagnosis relies on subjective questionnaires, and there is currently no effective solution to the often-convoluted process of depression diagnosis and treatment. The DSM-5 is the basis for self-reporting used for depression diagnosis, such as the PHQ-9 or its predecessors, BDI and HAM-D, and no biological diagnostic tests for MDD, such as blood work or MRI, are available on the market. However, these self-reporting instruments have multiple flaws, including measurement invariance issues and the assumption that depression symptoms can be summed up into a single score.

For example, psychometric analysis shows that measurement invariance (the idea that scales measure the same construct across groups) may not be true for the most popular scales. Another problem is that self-reports usually assume that you can add up symptoms to one summed depression “score”. However, neurophysiological studies (e.g., Drysdale et al., 2017) advocate for the identification and use of distinct depression subtypes, both for accurate depression diagnostics and in order to choose adequate treatment. Therefore, a need exists for improving screen of drug trial participants involved in the testing of pharmaceutical medications.

By leveraging the power of artificial intelligence and machine learning, the Brianify.AI can accurately predict the likelihood of an individual responding to a placebo, enabling the enrollment of only placebo non-responders in trials. This targeted approach increases the chances of achieving positive trial outcomes and accelerates the development of effective treatments for various psychiatric disorders.

Further, the Brianify.AI's capabilities extend beyond depression, as its transfer learning techniques allow for the rapid development of Placebo Non-Responders models for a wide range of other diseases. This adaptability makes Brianify.AI as a versatile solution in the field of clinical research, with the potential to revolutionize the drug development process across multiple therapeutic areas.

Further, the Brianify.AI enhances a clinical trial outcome by:
1. Streamlining trial design and reducing costs by identifying placebo responders at the beginning of the trial phase.
2. Utilizing advanced AI and machine learning models to accurately predict the probability of an individual responding to a placebo.
3. Excluding potential placebo responders from trials, increasing the likelihood of positive trial outcomes and accelerating the drug development process.
4. Adapting to a variety of diseases through transfer learning techniques, making it a versatile solution for clinical research across multiple therapeutic areas.

PlaceboInsight AI's capabilities extend beyond depression, as its transfer learning techniques allow for the rapid development of placebo response prediction models for a wide range of other psychiatric diseases. This adaptability makes PlaceboInsight AI a versatile solution in the field of clinical research, with the potential to revolutionize the drug development process across multiple therapeutic areas. In summary, PlaceboInsight AI offers a comprehensive solution for enhancing clinical trial outcomes using advanced AI and machine learning models by:
1. Identifying placebo responders based on the subjects' EEG recordings captured at screening or baseline visit (i.e. before randomization).
2. Predicting the probability of an individual subject responding to a placebo.
3. Predicting the prognostic score (or the score percentage change from baseline) if the subject were to receive placebo.
4. Increasing the statistical power of the clinical trial, increasing the likelihood of positive trial outcomes, and accelerating the drug development process.
5. Adapting to a variety of diseases through transfer learning techniques, making it a versatile solution for clinical research across multiple therapeutic areas.

PlaceboInsight AI has the potential to transform the landscape of clinical trials and expedite the development of effective treatments for psychiatric disorders and beyond, ultimately improving the lives of hundreds millions of people worldwide.

TherapyInsight AI is a groundbreaking product developed by Brainify.AI to revolutionize the clinical trial process and patient care in psychiatry. It is designed to identify subjects who are most likely to respond to new therapeutics, thereby improving the overall efficiency and success rate of clinical trials. In patient care, TherapyInsight AI will serve as a companion diagnostic test to predict a patient's response to the treatment. This personalized approach to treatment prediction has the potential to drastically change the landscape of drug development, ultimately leading to more effective treatments for patients suffering from psychiatric disorders.

TherapyInsight AI model for treatment response prediction is initially built using data and results from phase 2 clinical trials. To create these tailored tests, TherapyInsight AI employs cutting-edge transfer learning technology, which is applied to a range of machine learning (ML) models specifically built for treatment response prediction, such as Brain Age Prediction, Sex Prediction, and clustering models. By leveraging these ML models and integrating them with phase 2 trial data, TherapyInsight AI can accurately predict treatment response and identify the most suitable candidates for phase 3 trials.

In essence, TherapyInsight AI is a product used for biomarker identification for treatment response prediction. It achieves this by utilizing already built models and applying transfer learning to the dataset obtained from phase 2 trials. This innovative approach streamlines the clinical trial process, reducing the time and resources required to bring new treatments to market. In a clinical setting, TherapyInsight AI will serve as a companion diagnostic test to predict a patient's response to a particular treatment, thus assisting healthcare professionals in selecting the most suitable treatment option.

TherapyInsight AI offers numerous benefits to pharmaceutical companies, researchers and patients alike:
1. Precision: By utilizing ML models and transfer learning, Brianify.AI can accurately predict an individual's response to specific treatments, ensuring that only the most suitable candidates are enrolled in phase 3 trials. This precision increases the likelihood of positive trial outcomes and helps advance the development of effective therapeutics.
2. Efficiency: The Brianify.AI streamlines the clinical trial process by identifying the most promising treatment candidates early in the trial process. This reduces the time and resources required for trial completion, allowing for faster development and approval of new therapies.
3. Personalization: The Brianify.AI's ability to identify and predict individual responses to treatments ensures a more personalized approach to drug development. This personalized approach has the potential to improve treatment outcomes for patients and lead to the development of more targeted therapies.
4. Adaptability: The transfer learning technology employed by the Brianify.AI enables the rapid adaptation of ML models to different diseases and conditions. This adaptability makes the Brianify.AI a versatile solution for clinical research across multiple therapeutic areas.
5. Innovation: The Brianify.AI AI represents a significant advancement in the field of clinical research, combining advanced ML models, transfer learning technology, and personalized treatment prediction to revolutionize the drug development process.

Further, the Brianify.AI address the challenges associated with using electroencephalogram (EEG) data in machine learning (ML) models for the study of neurological and psychiatric disorders. Researchers often rely on ML models to analyze anatomical and functional neuroimaging data, including EEG, to generate diagnostic assessments and predict outcomes. While EEG data is relatively inexpensive and easy to collect, it requires accurate and efficient harmonization and preprocessing to achieve meaningful results from ML models. A major obstacle in the widespread adoption of EEG-based ML models for both basic research and translational purposes is the lack of standardized approaches to harmonize EEG data collected from multiple sources. This absence leads to two significant issues: investigators using “home-brew” solutions to merge non-uniform EEG data and the presence of irrelevant signals that cause data leakage, compromising the algorithm's prediction accuracy.

Further, the Brianify.AI addresses these challenges by providing a comprehensive solution that enhances the quality of EEG data and ensures its suitability for ML applications. Further, the Brianify.AI is designed to harmonize EEG datasets from different sources and prevent data leakage, ultimately increasing the accuracy and utility of predictive ML models. By employing cutting-edge ML techniques, the Brianify.AI effectively preserves biologically relevant EEG signals while eliminating unwanted noise. As a result, it enables researchers to obtain meaningful insights from their ML models, which can lead to improved diagnostic assessments and treatment predictions for various neurological and psychiatric disorders.

Further, the Brainify.AI is designed to be flexible and adaptable to meet the needs of specific clinical trials. The Brainify.AI use a transfer learning approach to refine machine learning models to the unique characteristics of each trial.

### Data Collection During Pharma Clinical Trial:

To generate predictions from Brainify.AI's models, EEG recordings will be required for each patient. These recordings will include a resting state condition and a number of short tests, developed according to the RDOC framework. Additionally, patients will need to fill in widely-used depression questionnaires such as HDRS-17 and QIDS. The entire process will take approximately 60-90 minutes. Brainify.AI can work with all widely-used EEG formats and layouts, so there are no specific technical requirements for choosing an EEG device for recording. In order to predict treatment response for new therapeutics, Pharma will need to collect additional data. The main component of this data will be several ERP tasks that coverall RDOC domains, as well as additional questionnaires such as MADRS, PHQ-9, WSAS, PSQI, GAD-7, and SHAPS. These data will be administered at different stages of the clinical trial (baseline, middle of the trial, and follow-up) and will be used for more detailed feedback and monitoring of the patient's response to treatment. Collecting this additional data will increase the overall time of data collection to approximately 200 minutes.

### Dataset Integration:

Machine Learning (ML) is widely being used to study neurological diseases and prediction of treatment outcomes. ML has been utilized to analyze electroencephalogram (EEG) datasets to identify prospective physiological biomarkers of psychiatric, neurological, and neurodevelopmental disorders. To ensure accuracy, ML algorithms must be trained on large datasets. However, in order to create EEG datasets that are large enough for training such a model, data sets must be consolidated across multiple patients, sites, and studies. There are currently no widely-implemented or standardized approaches for unifying EEG data from multiple sources. With the exception of the UK Biobank4, there is no unified EEG repository in the U.S., the EU, or any other country.

The absence of standardized and easily implemented protocols to combine EEG datasets creates two main problems. First, individual teams of researchers must overcome the challenges of merging data collected with different layouts and non-uniform formats produced by different devices (e.g., different numbers of electrodes). Although the research community puts forward protocols to case EEG data sharing (e.g., EEG-BIDS), following these protocols requires considerable computational skills not available to all research teams. Second, training ML models on datasets can result in data leakage. Data leakage occurs when an ML model acquires spurious information that compromises the accuracy of the algorithm's predictions. Notably, EEG datasets contain non-physiological features from recording equipment or location-specific phenomena that, if unaddressed, can lead to data leakage. While not widely discussed in the neuroscience community, a recent review of studies analyzing neuroimaging data with ML found evidence of data leakage in 46% of cases. For EEG data with variations in signal-to-noise ratios across sources, reducing data leakage is especially challenging, but essential to train models to make accurate predictions.

To overcome these barriers, Brainify.AI is developing an all-in-one data pipeline to merge EEG data from multiple sources and eliminate data leakage. Unlike publicly available EEG preprocessing pipelines, “EEG Data Whitening Model” combines EEG datasets using state-of-the-art ML algorithms to eliminate data leakage sources. Brainify.AI's preliminary work includes development of a deep convolutional neural network (DCNN) model capable of identifying data leakage from site-and equipment-related noise. In a preliminary study, model used data leakage to predict the exact location of EEG recordings across multiple sites with 92% accuracy. This accuracy was preserved even after controlling for sample demographic characteristics and using advanced noise removal approaches.

Recently, Brainify.AI improved their model by introducing a Generative Adversarial Network (GAN) to perform unsupervised noise identification and removal. This approach removed noise such that DCNN site prediction accuracy was reduced by 15%. In this project, Brainify.AI will incorporate this ML model into a pipeline that also preprocesses, labels, and differentiates EEG data. Addressing this need will facilitate training of ML models to identify physiological disease biomarkers and predict treatment outcome. Brainify.AI's EEG Data Whitening Model developed and tested using publicly and non-publicly available EEG datasets. Dataset has been assembled with various cap layouts obtained by subsetting the international 10/5 system and the geodesic system for electrode placement. Data are presented in various modalities: the resting state with open and closed eyes and task-related EEGs for more than 20 different tasks. The dataset includes descriptive metadata, which comprises more than 1700 different subject-and session-level attributes.

### Data Processing and Analysis:

The Brainify.AI platform uses the most advanced EEG data processing techniques to ensure the high quality and the reliability of the Brainify.AI biomarkers. The first step of analysis is EEG preprocessing that uses automatic artifact removal based on the statistical properties of EEG, data standardization and ML-based data transformation to harmonize data from different sources and reduce data leakage problem.

Based on the generative embedding approach, pre-processed EEG is used to calculate pre-selected quantitative EEG (qEEG) features that will then inform the architecture of the developing deep convolutional neural networks (DCNN) that will use the EEG signal directly. For the pre-selected feature generation, more than 4000 qEEG characteristics has been calculated to develop the best architecture of the DCNN models for precise and accurate calculation of the biomarker scores and prediction the level of the placebo response. Further, the calculated qEEG features is used for deeper understanding and explain ability of the DCNN models. There are several challenges in EEG data analysis that Brainify.AI platform was designed to overcome. The first one is stable and fast automatic artifact removal.

Further, the EEG Data Whitening model uses GAN and CNN ML models to harmonize data and avoid data contamination. The EEG Data Whitening model is used in addition to the automatic artifact removal techniques to ensure that non-physiological noise is removed from EEG and does not affect the identification of biomarkers. It also gives the possibility to combine EEG recorded with any type of EEG device, which is crucial for building large-scale datasets that are crucial for the advances in biomarker studies. The last challenge for EEG analysis is a significant inter-individual variability in peoples' brain activity which, together with the low signal-to-noise ratio, makes it hard to achieve high accuracy of predictions from EEG.

Further, the Brainify.AI addresses the last challenge by using state-of-the-art deep convolutional neural network models optimized for EEG data analysis.

### Technical Architecture:

1. Data ingestion:
   First layer of the system is the data ingestion layer. Further, the data ingestion includes stream and batch data ingestion. Further, the system may support next formats of EEG data:
      Brain Vision (.vhdr, .vmrk, .eeg)
      European data format (.edf)
      BioSemi data format (.bdf)
      Curry (.cdt)
      General data format (.gdf)
      Neuroscan CNT (.cnt)
      EGI simple binary (.egi)
      EGI MFF (.m)
      EEGLAB files (.set, .fdt)
      Nicolet (.data)
      EXimia EEG data (.nxe)
      Persyst EEG data (.lay, .dat)
      Nihon Kohden EEG data (.eeg, .2le, .pnt, .log)
      XDF data (.xdf, .xdfz)
   Real-time Stream Ingestion relies on a message queue which serves as the backbone for low-latency insertion of events, such as uploading new EEG data, storing the events for a set amount of time, and delivering them downstream for real-time processing. The message queue offers scalability and durability, enabling it to withstand zone or region outages. Restful interface precedes the message queue, providing the agility to connect new clients through preferred protocols rather than specific tools. Incoming events from the message queue are handled by the stream processing service on an event or micro-batch basis. Batch Ingestion is a crucial component of data pipeline, to efficiently load and process large datasets or collect data uploaded to the system on a scheduled regular basis. This service is particularly useful for clinical trials and other research studies that generate a significant amount of data.
   In contrast, Real-Time Stream Ingestion is designed for low-latency insertion of events, such as uploading new EEG data, and providing real-time processing. This service relies on a message queue and RESTful interface, which allows for scalability and durability, ensuring the system can withstand zone or region outages. Both the stream and batch processing services upload data to data lake, where it is securely stored and processed for downstream analytics and machine learning models. This ensures that Brainify.AI can handle a high volume of data while providing accurate and timely insights for customers.
2. Data lake
   A scalable and highly-durable storage service that supports features like storage classes (differentiation between hot and cold storage), object lifecycle management (automatically change storage class after a specified time period), versioning, encryption-at-rest and fine-grained security policies.
3. Preprocessing pipeline
   This layer provides validation and preprocessing services. The data validation services assess each EEG data file according to a set of rules (such as checking for bad or bridging electrodes, amplitude outliers, and others). The resulting data quality report helps automatically reject poorly acquired data. The preprocessing service runs the data files through a set of filtering, detection, and artifact removal steps, and converts the data to a unified format that can be used by the analytical platform. From a technical perspective, this involves a number of Docker containers that run on the Kubernetes cluster.
4. Analytical Platform
   The Models Development part of the Analytical Platform is where neuroscientists and data scientists can develop, train, and validate models based on DCNN and CNN. Once the model is ready for production, it is deployed to a scalable and durable Kubernetes cluster.
5. Reports
   Reporting layer of the system provides set of the reports such as:
      Placebo non-responders report
      Treatment responds report
      Data quality report.
6. Central services
   This set of cross-functional services supports the daily operational tasks of the system. The Security & Audit service manages user groups and access rights, and provides security and access logs and reports. The Process & Cost Monitoring & Logging service offers various runtime logs and system health support services, including online alerts, log management, and reporting. The Continuous Deploy & Integration service provides development and deployment-oriented services such as source control, continuous integration, and continuous deployment. The Automation & Scheduling service enables the automation and orchestration of system behavior based on events or schedules.
   Examples:
      Service numbers calling according to load
      Reports generation
      Batch processing scheduling

### Machine Learning Models:

1. EEG Data-Whitening Model:
   EEG data are quite noisy and can be affected by patients' movements, lab settings, clinicians' mistakes, and electroencephalographs. It is common practice to clean EEG data of potential interference outside of brain activity before using them. Examples of sources of such unwanted signals are eye movements (EOG), heartbeat (ECG), involuntary muscle activity (EMG), and interference from the power supply network (50 Hz or 60 Hz). Also, to prepare EEG data, it is necessary to exclude the influence of the unevenness of the frequency response of the entire EEG data acquisition path.
   The structure of the skull, the properties of the skin of the subject's head, the quality of the electrode gel, the material and quality of the electrodes, and the density of contact with the skin are some of the possible causes of uneven frequency response. Wires and preamps can also introduce distortion.
   The Brainify.AI has developed an original EEG Data-Whitening Model, an EEG preprocessing pipeline that aims to clear data of most of the negative phenomena. Further, the Brainify.AI use bandpass, notch, and median filters with average electrode re-referencing, followed by independent interquartile range normalization for each channel and EOG correction based on wavelet transformations. Further, the Brainify.AI utilize frequency response whitening, which normalizes frequency spectra over an EEG block for each channel and converts the data back to the time/amplitude domain. The final stage consists of another interquartile range normalization for each channel and removal of amplitude outliers.
   EEG Data Whitening Model based on the following technical objectives:
      EEG Quality Control Checker (QCC): QCC pipeline streamline the process of combining EEG data in multi-site studies. This library used to create an automatic QCC that merge and return quality statistics of multi-site data.
      Reduce EEG data leakage: ML model reduced data leakage-dependent site prediction accuracy by 15%. ML tool identifies data leakage in EEG signals and removes non-physiological noise. Usage of data collected from a single study conducted at different sites and ensure that source specific dataset characteristics are not biasing model learning.
   Preserve clinically-relevant physiological signals: To ensure valid, clinically-relevant physiological signals are preserved post-transformation, assess the accuracy published DCNN models to predict age from EEG activity. Additional checks of quantitative EEG (qEEG) features (amplitude, frequency, and entropy) performed to verify data transformations do not affect relevant characteristics of the recorded brain activity.
   As a result, the EEG Data-Whitening Model:
      Removes subject-related artifacts, such as eye blinking, eye movements, muscle tension, and body movements
      Removes device-related and laboratory environment-related artifacts
      Makes Brainify.AI models and algorithms compatible with EEGs recorded with a variety of devices on the market
      Has been tested on 15,000 unique EEG records.
2. Cluster-based Model:
   Clustering is a data-driven technique used in machine learning to identify groups of people with close characteristics. The method projects EEG signals of a patient to a 128-dimensional space, where separate patient subgroups can be found. Each patient is represented as a value in a latent multi-dimensional space where clusters of patients can be discovered by ML algorithms based on their proximity to each other. This method can be used for identification of both individual stable EEG characteristics (“EEG fingerprints”), and for finding more dynamic EEG patterns associated with different brain states or changes in the brain due to disorders. The clustering-based model is expected to identify patient subgroups corresponding to healthy controls or depression subtypes in the constructed 128-dimensional space. The obtained information is later used for differential treatment response prediction.
3. Placebo Non-Responders Model:
   The gold standard for testing any kind of intervention is the clinical trial that is randomized, double-blind, and placebo-controlled. A placebo is a “sham” intervention that is designed to have no treatment effect. Despite the fact, patients that are subjected to placebos in clinical studies often do show responses, in what is known as the placebo effect. The effect is particularly noticeable in depression studies, with it being one of the main causes for the significant number of failures in the development of novel antidepressant treatments. Specifically, high placebo-response rates reduce the rates of positive outcomes in phase II and III clinical trials. In order to speed up the process of phase II clinical trials and increase the statistical power of such studies, it is proposed that trial volunteers who are likely to respond to a placebo be excluded from such studies, and this decision is likely to yield results that are beneficial to the researchers and pharmaceutical companies involved.
   The individual characteristics of placebo non-responders, specifically for depression patients, were identified in studies by advisor, Diego Pizzagalli, on a group level (Trivedi et al., 2018; Ang et al., 2022). Some of these characteristics were related to the general state of the patient (e.g., a younger age, high cognitive processing speeds, or an absence of a history of physical abuse). Other characteristics were those hypothesized to be related to specific subtypes of depression (as characterized by factors such as levels of anxiety or anhedonia). Independently, it was shown that the probability that an individual will respond to a placebo can be predicted from their EEG resting state (also supported by a recent meta-analysis by Whatts et al., 2022). The predictive EEG characteristics include both the more basic quantitative features of the signal (e.g., higher rostral theta current density) and the more sophisticated EEG functional connectivity measures (i.e., greater alpha-band and lower gamma-band connectivity-most prominently parietal).
   The placebo Non-Responders AI/ML model can predict and score individual placebo-response probabilities. Based on team previous work, developed the Placebo Non-Responders AI/ML model, using the most up-to-date EEG placebo dataset from the EMBARC study. This model has the ability to predict and score individual placebo-response probabilities, utilizing pre-selected feature generation and data-driven deep neural network algorithms. The generative embedding approach employed in the model allows for highly accurate scores of an individual's probability of responding to a placebo, with the highest score of 86% achieved based on the model's area under the curve metric. This model is capable of working on both behavioral+EEG and EEG-only features. During Phase II and III clinical trials, these scores will be used to segment participants, ensuring that those with high probabilities of responding to placebos are not enrolled in studies. This improves the likelihood of positive outcomes for clinical trials. Although the model was initially based on depression patients, the AI's transfer learning techniques can be applied for the rapid development of versions of Placebo Non-Responders models for a variety of other diseases.
   In summary, the Placebo Non-Responders Model:
      Identifies the probability that an individual will respond (or will not respond) to a placebo
      Segments participants using easy-to-calculate measures
      Allows the enrollment of only placebo non-responders in clinical trials to increase the chances of positive trial outcomes.
4. Brain Sex Phenotype Prediction Model:
   Converging evidence points to a relationship between fluctuations in ovarian hormone levels and symptoms of depression in females. First, sex differences related to depression emerge in adolescence and decline during post menopause. Second, the greatest chances of experiencing depression are associated with periods of maximal change in ovarian hormone levels (e.g., during and after pregnancy or during the menopausal transition). Third, females tend to experience some changes in affective symptoms according to the menstrual cycle (with the possibility of an unfavorable outcome, such as premenstrual dysphoric disorder). Fourth, recent studies have demonstrated that oral contraceptives (which affect hormonal regulation) are associated with an increased risk of developing depression.
   At the same time, males and females have different responses to and outcomes from antidepressant therapies. Females exhibit a better response to serotonin reuptake inhibitors (SSRIs) and to monoamine oxidase inhibitors (MAOIs), while males respond more positively to tricyclic antidepressants. This difference has been attributed to the effects of estrogen. Previous research in psychopathology and sex differences was based on a binary view of sex (meaning that female and male brains were considered either exactly the same or totally different). Further, the Brainify.AI will automatically analyze EEG data to aid in depression diagnostics, which works toward the ultimate goal of developing ML-based approaches to guide treatment decision-making.
   Overall, the Brainify.AI will lead to new, automatic, highly accurate, and scalable methods for identifying female hormonal and “brain sex phenotype-related” depression. The automatic identification of depression subtypes will help to overcome the traditional clinical diagnosis problem 38 (based on subjective questionnaires) that arises from treating patients as a unified group when that is not the case.
5. Brain Age Prediction Model:
   There are multiple age-related changes in the brain. At a certain point around 65 years of age, a gradual decline in connectivity between distinct brain regions is observed in normal ageing; in turn, numerous cognitive dysfunctions begin to appear. Some people with mental health conditions are more prone to experiencing the early onset of these cognitive dysfunctions. Recent studies have confirmed an existing variability between chronological age and accelerated brain aging in those with mental disorders and early life stress. These findings have led to a hypothesis that the age of the brain may serve as a biomarker for the early diagnosis of certain mental and neurodegenerative conditions.
   A combination of the Brain Sex Phenotype Prediction Model and the Brain Age Prediction Model is crucial for disentangling different female depression subtypes. As a result, the Brain Age Prediction Model:
      Identifies age from EEG data with high accuracy
      Captures accelerated aging-related changes in the brain
      Disentangles age-related depression from other subtypes of depression
      Assists in choosing effective treatment for age-related depression.

### Validation Strategy for Machine Learning Models:

The validation strategy for machine learning models is critical to ensuring the accuracy and reliability of predictions. A multi-step approach to validate models, including both internal and external validation methods.

Internal validation methods:
1. Cross-validation:
   Models are trained on a portion of the data and then tested on the remaining data to assess their performance. This method allows to determine if models are overfitting to the training data. To correctly assess the model quality, 10-fold cross-subject cross-validation with separate validation and testing datasets are used. The cross-validation procedure was repeated ten times. At each iteration, the whole dataset was divided into ten parts, whereby eight parts were used for training the network, one for validation during training, and one for testing the final model. Eye open and eye closed EEG session segments corresponding to the same subject in the same fold is used to detect patterns among different EEG recordings and not memorize sessions.
2. Bootstrapping: Sample the data multiple times to create multiple training sets. This allows to evaluate the stability of models and ensure that they are not overfitting to specific patterns in the data.

### External Validation Methods:

1. Independent dataset validation: Models are tested on an independent dataset to assess their generalizability to new data. This dataset should be representative of the population that aimed to make predictions for.
2. Collaborative validation: Validation is based on academic and research institutions data. This allows to assess the performance of the models in real-world settings and ensure that they are accurately predicting treatment and placebo responses.

Further, the system may integrate advanced artificial intelligence models, such as deep neural networks or convolutional neural networks, to analyze EEG patterns. These models can be trained on historical data to predict treatment responses with high accuracy, thereby improving clinical decision-making. Further, implementation involves feeding raw EEG data into AI models, which are then optimized using techniques like transfer learning to generalize across different datasets.

Further, the system may fuse EEG data with additional patient data sources, such as clinical history, genetic information, or imaging results. This fusion enhances prediction accuracy by capturing a broader range of factors influencing treatment response. Further, implementation involves creating integrated datasets and employing fusion techniques like attention mechanisms in neural networks to weigh the importance of different data types.

Further, the system may be integrated with wearable devices to collect continuous EEG data for providing a more comprehensive view of patient states over time. These devices can also monitor other physiological metrics, offering a richer dataset for analysis.

Further, the system may adapt clinical trial designs based on real-time predictions of treatment response. This allows for dynamic adjustment of trial parameters, reducing recruitment challenges and shortening trial durations. Further, the implementation may involve creating adaptive algorithms that adjust inclusion criteria and treatment arms based on predicted outcomes.

Further, the system may utilize real-time AI models to stratify patients based on predicted treatment responses. This allows for immediate identification of suitable candidates for specific clinical trials, optimizing resource allocation and trial efficiency. Further, the implementation involves deploying machine learning models in real-time processing pipelines.

Further, the system may include neural networks designed to process not only EEG data but also imaging or genetic information, providing a more comprehensive understanding of treatment mechanisms. This approach enhances the depth and accuracy of predictions. Further, implementation involves constructing architecture that combine diverse data types through fusion layers.

Further, the system may leverage predictive analytics to forecast the likelihood of treatment success at various stages of therapy. This enables early intervention and adjustment of treatment plans as needed. Further, implementation involves developing forecasting models that account for dynamic patient changes.

Further, the system may aggregate contextual data from various sources, such as environmental factors or lifestyle information, to refine treatment predictions. Further, the implementation involves collecting and integrating diverse contextual data points into analysis models.

Further, the user may customize prediction models based on specific clinical needs or patient demographics. This flexibility allows for tailored solutions across different therapeutic areas. Further, the implementation involves creating model architecture that support easy customization through user interfaces and modifiable parameters.

Further, the system may include a multilayer training architecture for machine learning models, specifically designed for predicting patient response to depression treatment. Further, the first layer may include a masked variational autoencoder (VAE) to leverage unlabeled EEG records. Further, the first layer may utilize pre-trained models or transfer learning approaches to leverage knowledge from other related tasks or domains. This can help reduce overfitting and improve generalization.

Further, the second layer may include a conditional masked variational autoencoder (CMAE). Further, the second layer may implement model pruning techniques to reduce the number of parameters in the CMAE model, improving efficiency and reducing the risk of overfitting. Further, the second layer may utilize knowledge distillation approaches to transfer knowledge from a larger teacher network to a smaller student network, enabling more efficient inference.

Further, the third layer may implement class weighting techniques to address class imbalance issues and improve model performance. Further, the third layer may utilize ensemble methods, such as bagging or boosting, to combine predictions from multiple models and improve overall accuracy.

Further, the fourth layer may implement regularization technique such as dropout or L1/L2 regularization, to reduce overfitting and improve generalization.

Further, the system may combine multiple models trained on different subsets of data or with varying hyper parameters to improve overall performance and robustness.

Further, the system may utilize attention mechanism to focus on relevant features or regions of interest in the EEG data. Further, the system may utilize graph neural networks to leverage graph-structured data and improve performance on tasks such as subject clustering and sex & age prediction.

Further, the system may utilize reinforcement learning framework to training the multilayer training architecture.

Further, the system may incorporate advanced signal processing techniques such as independent component analysis (ICA), an empirical mode decomposition (EMD), and sparse representation-based methods can further enhance the quality and accuracy of EEG data analysis.

Further, the Deep Convolutional Neural Network (DCNN) of the system may be tailored to accommodate specific types of EEG data, such as resting-state or ERP sessions, can improve its ability to capture relevant features and patterns.

Further, the system may utilize pre-trained DCNN models trained on large datasets. Further, the system may adapt them for EEG-based tasks using techniques like transfer learning and knowledge distillation. Further, the incorporation of both self-supervised (e.g., autoencoder) and supervised learning techniques into the algorithm can allow the system to leverage both labeled and unlabeled data, leading to improved robustness and generalizability.

Further, the system may implement augmentation techniques or generate synthetic EEG data through methods like Generative Adversarial Networks (GANs) to expand the dataset's size and diversity. Further, the system may enhance the algorithm's ability to generalize across different subjects and scenarios.

Further, the system may develop a real-time processing module within the algorithm to provide immediate feedback and analysis during EEG data acquisition, allowing for more efficient and adaptive research protocols.

Further, the system may integrate user-friendly visualization tools and reporting features into the algorithm to facilitate easier interpretation of results by researchers and clinicians, promoting broader adoption and application in various fields.

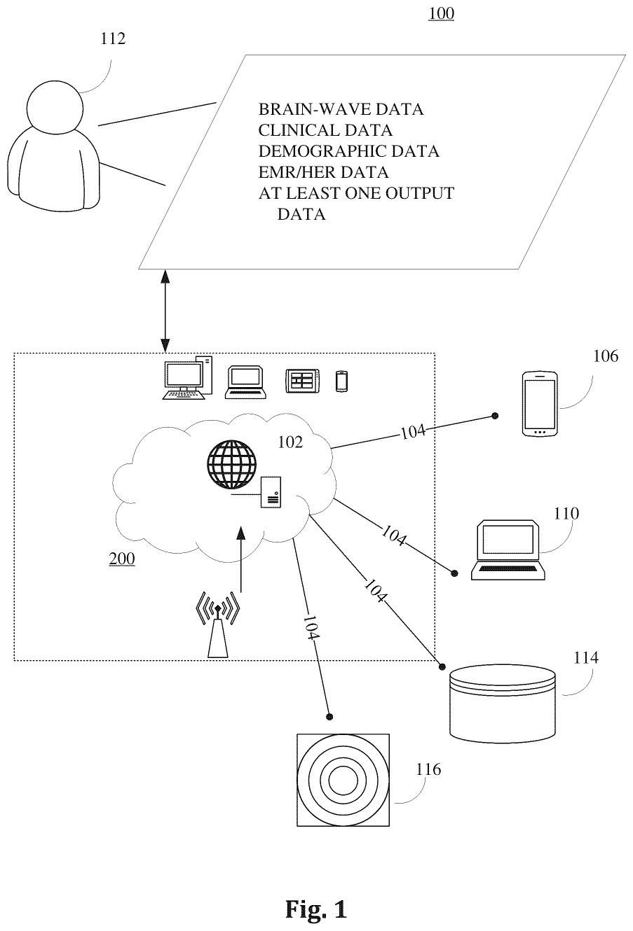

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

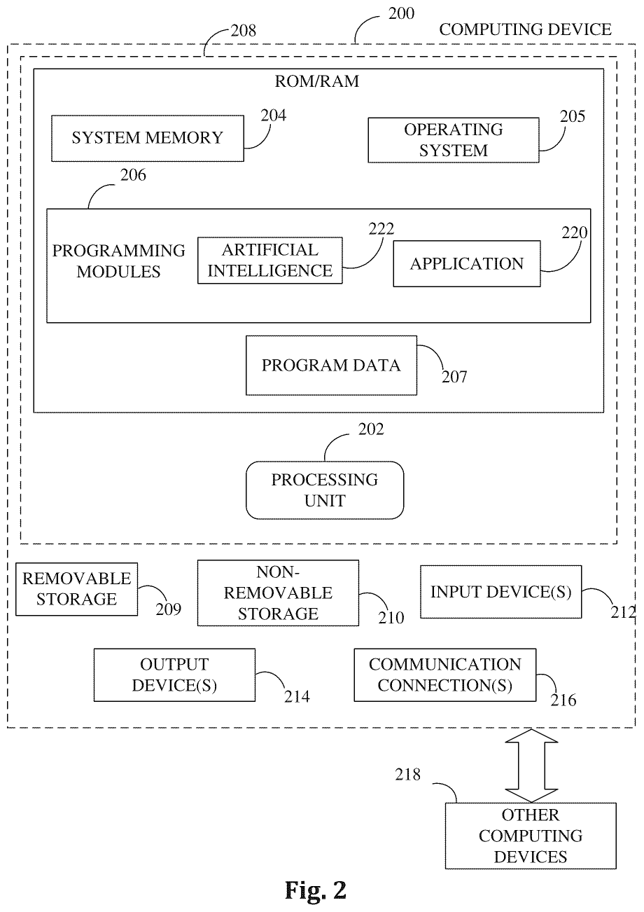

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player and artificial intelligence 222) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

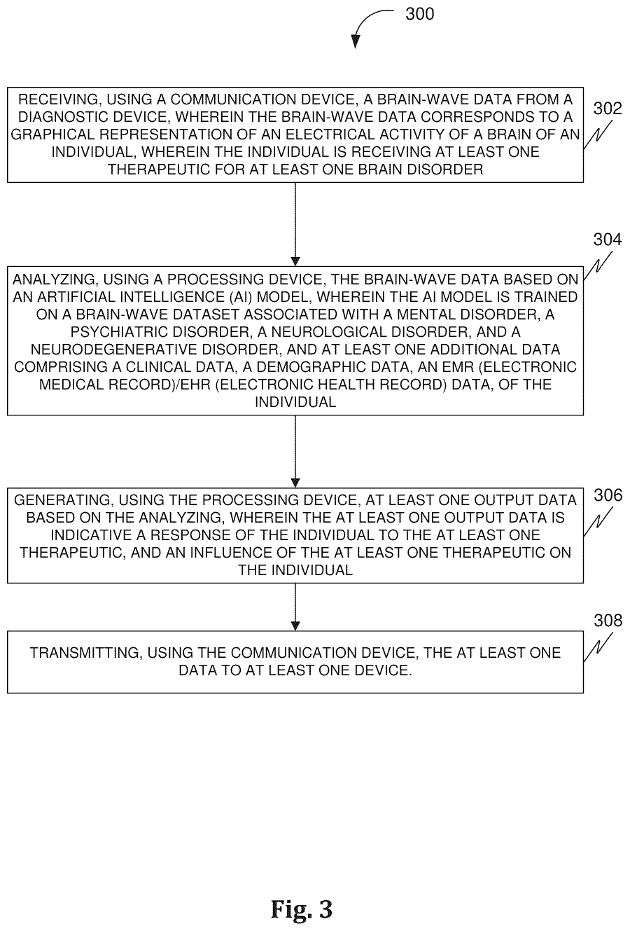

FIG. 3 illustrates a flowchart of a method 300 of EEG-based biomarker discovery and commercialization for personalized diagnosis and treatment of brain disorders, in accordance with some embodiments.

Accordingly, the method 300 may include a step 302 of receiving, using a communication device 702, a brain-wave data from a diagnostic device 802. Further, the diagnostic device 802 may include an electroencephalogram (EEG) capturing device. Further, the brain-wave data corresponds to a graphical representation of an electrical activity of a brain of an individual. Further, the individual may include a patient and a subject. Further, the brain-wave data may include an electroencephalogram (EEG) reading of the individual. Further, the individual may be in a resting state. Further, the individual may be undergoing an Event-Related Potential (ERP) session. Further, the individual may be receiving at least one therapeutic for at least one brain disorder. Further, the at least one therapeutic may include a mental therapy, a neurological therapy, a neurodegenerative therapy, and at least one drug. Further, the at least one brain disorder may include at least one of a mental condition, a psychiatric condition, a neurological condition, and a neurodegenerative condition. Further, the method 300 may include a step 304 of analyzing, using a processing device 704, the brain-wave data based on an artificial intelligence (AI) model. Further, the brain-wave data may be analyzed to identify one or more electroencephalogram (EEG) biomarkers. Further, the one or more EEG biomarkers may be indicative of a condition of the individual. Further, the one or more EEG biomarkers may facilitate monitoring of a condition of the individual receiving the at least one therapeutic. Further, the AI model may be trained on a brain-wave dataset associated with a mental disorder, a psychiatric disorder, a neurological disorder, and a neurodegenerative disorder, and at least one additional data comprising a clinical data, a demographic data, an EMR (electronic medical record)/EHR (electronic health record) data, of the individual. Further, the method 300 may include a step 306 of generating, using the processing device 704, at least one output data based on the analyzing. Further, the at least one output data may be indicative of a response of the individual to the at least one therapeutic, and an influence of the at least one therapeutic on the individual. Further, the method 300 may include a step 308 of transmitting, using the communication device 702, the at least one output data to at least one device.

Further, in an embodiment, the brain-wave data may include a raw brain-wave data. Further, the analyzing of the brain-wave data may include analyzing the raw brain-wave data based on the AI model. Further, the generating of the at least one output may be further based on the analyzing of the raw brain-wave data.

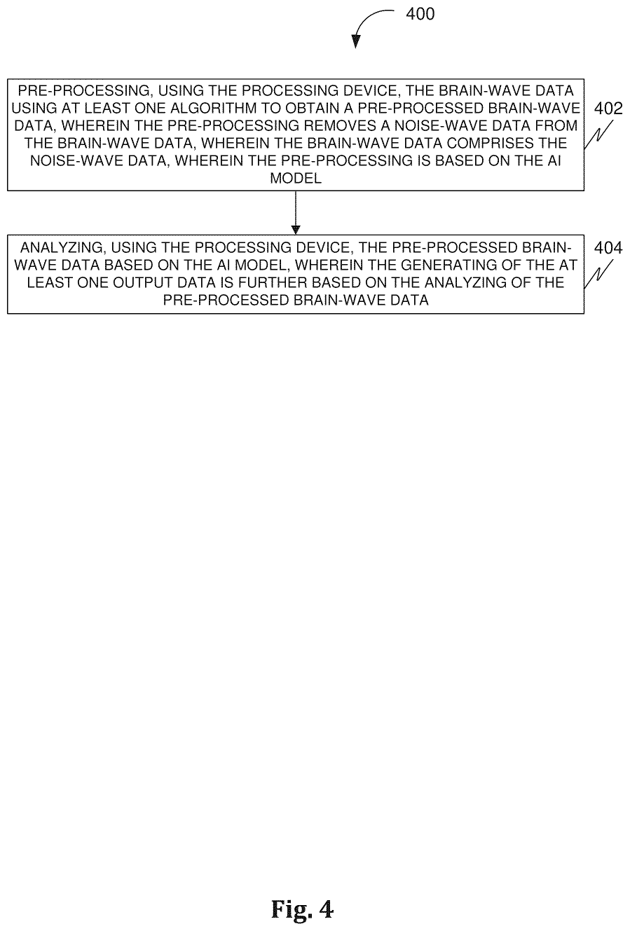

FIG. 4 illustrates a flowchart of a method 400 of EEG-based biomarker discovery and commercialization for personalized diagnosis and treatment of brain disorders including analyzing, using the processing device 704, the pre-processed brain-wave data, in accordance with some embodiments.

Further, in some embodiments, the method 400 further may include a step 402 of pre-processing, using the processing device 704, the brain-wave data using at least one algorithm to obtain a pre-processed brain-wave data. Further, the pre-processing removes a noise-wave data from the brain-wave data. Further, the brain-wave data includes the noise-wave data. Further, the pre-processing may be based on the AI model. Further, in some embodiments, the method 400 further may include a step 404 of analyzing, using the processing device 704, the pre-processed brain-wave data based on the AI model. Further, the generating of the at least one output data may be further based on the analyzing of the pre-processed brain-wave data.

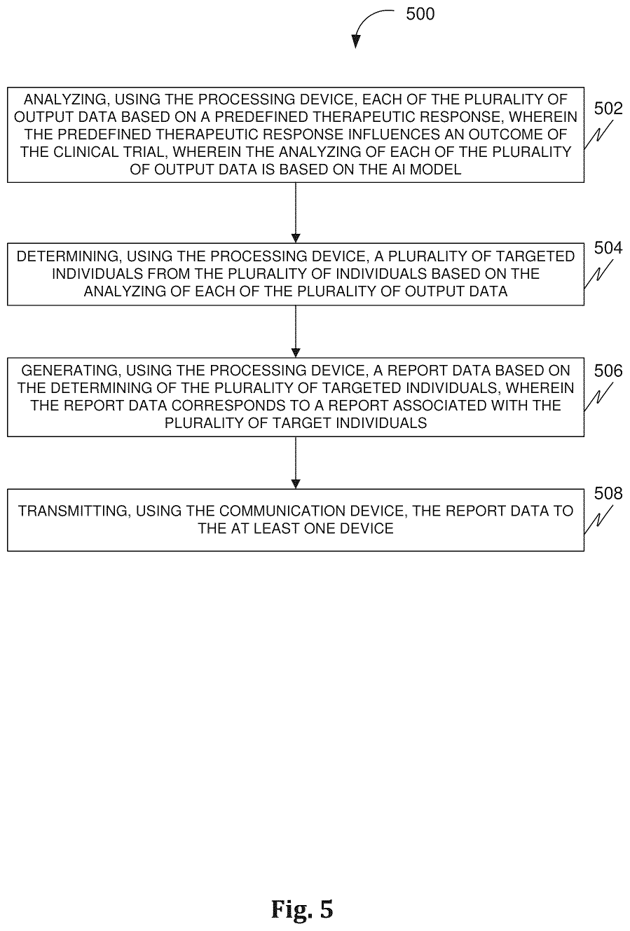

FIG. 5 illustrates a flowchart of a method 500 of EEG-based biomarker discovery and commercialization for personalized diagnosis and treatment of brain disorders including generating, using the processing device 704, a report data, in accordance with some embodiments.

Further, in some embodiments, the brain-wave data may include two or more brain-wave data corresponding to two or more individuals. Further, the at least one output data may include two or more output data corresponding to the two or more individuals. Further, the two or more individuals may be associated with a clinical trial of the at least one therapeutic. Further, the method 500 further may include a step 502 of analyzing, using the processing device 704, each of the two or more output data based on a predefined therapeutic response. Further, the predefined therapeutic response influences an outcome of the clinical trial. Further, the analyzing of each of the two or more output data may be based on the AI model. Further, the method 500 further may include a step 504 of determining, using the processing device 704, two or more targeted individuals from the two or more individuals based on the analyzing of each of the two or more output data. Further, the method 500 further may include a step 506 of generating, using the processing device 704, a report data based on the determining of the two or more targeted individuals. Further, the report data corresponds to a report associated with the two or more target individuals. Further, the method 500 further may include a step 508 of transmitting, using the communication device 702, the report data to the at least one device.

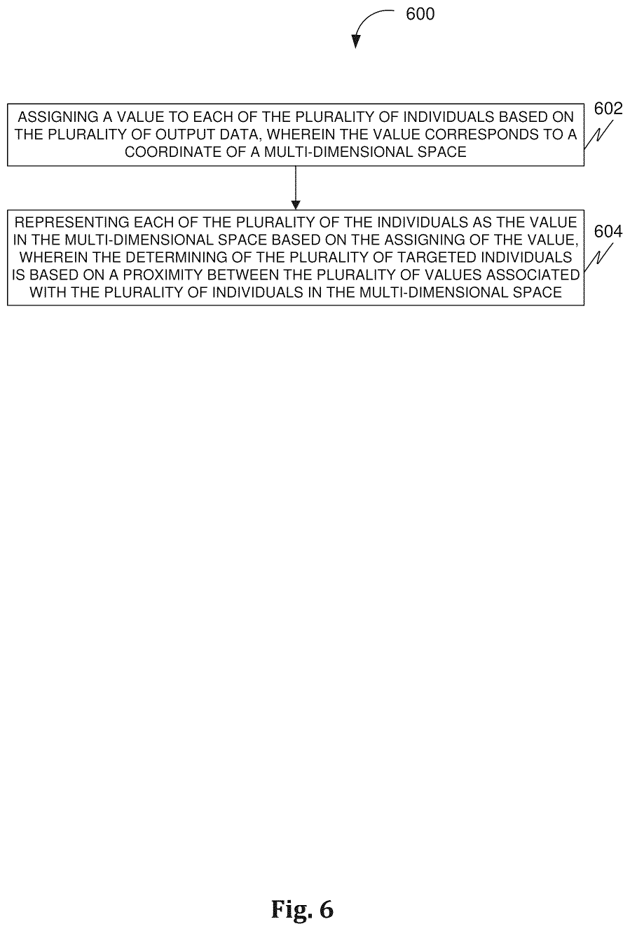

FIG. 6 illustrates a flowchart of a method 600 of EEG-based biomarker discovery and commercialization for personalized diagnosis and treatment of brain disorders including determining two or more targeted individuals, in accordance with some embodiments.

Further, in some embodiments, the analyzing of each of the two or more output data may include a step 602 of assigning a value to each of the two or more individuals based on the two or more output data. Further, the value corresponds to a coordinate of a multi-dimensional space. Further, the analyzing of each of the two or more output data may include a step 604 of representing each of the two or more individuals as the value in the multi-dimensional space based on the assigning of the value. Further, the determining of the two or more targeted individuals may be based on a proximity between two or more values associated with the two or more individuals in the multi-dimensional space.

In some embodiments, the two or more targeted individuals includes one or more of a placebo responder and a non-placebo responder. Further, the predefined therapeutic response includes one or more of a placebo response and a non-placebo response. Further, the placebo responder experiences a placebo-recovery from the at least one brain disorder. Further, the placebo-recovery may be associated with a placebo effect. Further, the non-placebo responder experiences a therapeutic-recovery from the at least one brain disorder. Further, the therapeutic-recovery may be associated with an active treatment of the at least one therapeutic. Further, the report includes one or more of a placebo responder report and a non-placebo responder report.

In some embodiments, the analyzing of the brain-wave data includes identifying a biological-characteristic of the individual. Further, the electrical activity of the brain may be based on the biological-characteristic. Further, the at least one brain disorder may be associated with the biological-characteristic. Further, the biomarker may be associated with the biological-characteristic.

In some embodiments, the brain-wave dataset includes each of a target-labelled brain-wave data and a target-unlabeled brain-wave data. Further, each of the target-labelled brain-wave data and the target-unlabeled brain-wave data may be recorded from two or more drug-trail participants. Further, the two or more drug-trail participants may be associated with a clinical trial of the at least one therapeutic comprising one or more of a placebo treatment and an active treatment. Further, the target-labelled brain-wave data includes an indicator indicating the at least one therapeutic associated with each of the two or more drug-trail participants. Further, the target-unlabeled brain-wave data lacks the indicator

In some embodiments, the AI model includes two or more AI models comprising each of the first AI model, a second AI model, a third AI model, and a fourth AI model. Further, the first AI model may be configured to be trained on a target-unlabeled brain-wave data based on a self-supervised learning technique. Further, the brain-wave dataset includes the target-unlabeled brain-wave data. Further, the second AI model may be configured for clustering two or more individuals in a multi-dimensional space. Further, the two or more individuals may be associated with the target-unlabeled brain-wave data.

In some embodiments, the second AI model may be further configured for predicting a biological characteristic associated with each of the two or more individuals. Further, the third AI model may be configured for predicting a response of the two or more individuals to a therapy. Further, the fourth AI model may be configured for predicting the therapeutic response of the two or more individuals to the at least one therapeutic. Further, the fourth AI model may be configured to be trained using a supervised learning.

In some embodiments, the AI model may be based on one or more of a deep convolutional neural network and a generative adversarial network.

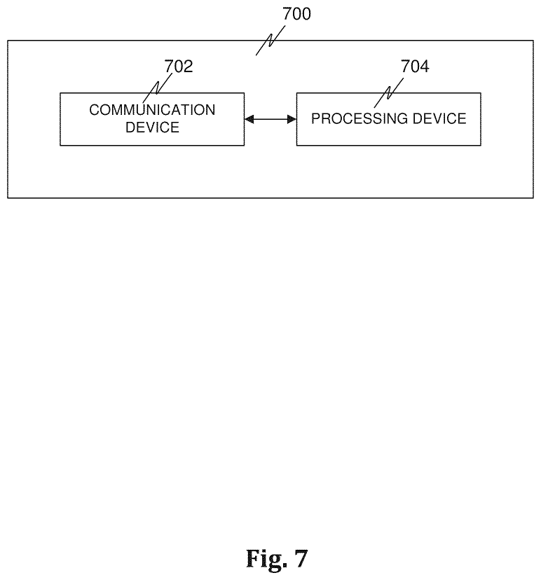

FIG. 7 illustrates a block diagram of a system 700 of EEG-based biomarker discovery and commercialization for personalized diagnosis and treatment of brain disorders, in accordance with some embodiments.

Accordingly, the system 700 may include a communication device 702. Further, the communication device 702 may be configured for receiving a brain-wave data from a diagnostic device 802. Further, the brain-wave data corresponds to a graphical representation of an electrical activity of a brain of an individual. Further, the individual may include a patient and a subject. Further, the brain-wave data may include an electroencephalogram (EEG) reading of the individual. Further, the individual may be in a resting state. Further, the individual may be undergoing an Event-Related Potential (ERP) session. Further, the individual may be receiving at least one therapeutic for at least one brain disorder. Further, the at least one therapeutic may include a mental therapy, a neurological therapy, a neurodegenerative therapy, and at least one drug. Further, the at least one brain disorder may include at least one of a mental condition, a psychiatric condition, a neurological condition, and a neurodegenerative condition. Further, the communication device 702 may be configured for transmitting at least one output data to at least one device. Further, the system 700 may include a processing device 704 communicatively coupled with the communication device 702. Further, the processing device 704 may be configured for analyzing the brain-wave data based on an artificial intelligence (AI) model. Further, the brain-wave data may be analyzed to identify one or more electroencephalogram (EEG) biomarkers. Further, the one or more EEG biomarkers may be indicative of a condition of the individual. Further, the one or more EEG biomarkers may facilitate monitoring of a condition of the individual receiving the at least one therapeutic. Further, the AI model may be trained on a brain-wave dataset associated with a mental disorder, a psychiatric disorder, a neurological disorder, and a neurodegenerative disorder, and at least one additional data comprising a clinical data, a demographic data, an EMR (electronic medical record)/EHR (electronic health record) data, of the individual. Further, the processing device 704 may be configured for generating the at least one output data based on the analyzing. Further, the at least one output data may be indicative of a response of the individual to the at least one therapeutic, and an influence of the at least one therapeutic on the individual.

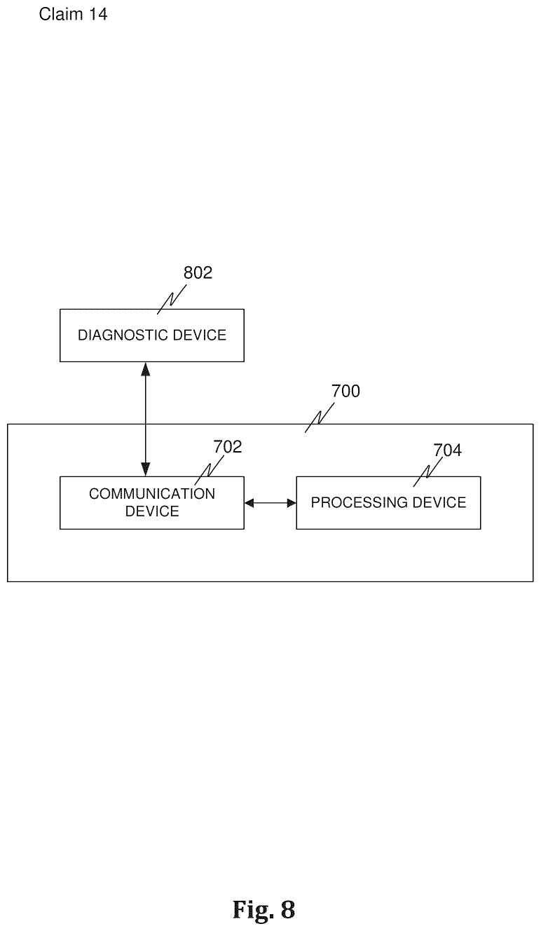

FIG. 8 illustrates a block diagram of the system 700, in accordance with some embodiments.

Further, in some embodiments, the processing device 704 may be further configured for pre-processing the brain-wave data using at least one algorithm to obtain a pre-processed brain-wave data. Further, the pre-processing removes a noise-wave data from the brain-wave data. Further, the brain-wave data includes the noise-wave data. Further, the pre-processing may be based on the AI model. Further, the processing device 704 may be further configured for analyzing the pre-processed brain-wave data based on the AI model. Further, the generating of the at least one output data may be further based on the analyzing of the pre-processed brain-wave data.

Further, in some embodiments, the brain-wave data may include two or more brain-wave data corresponding to two or more individuals. Further, the at least one output data may include two or more output data corresponding to the two or more individuals. Further, the two or more individuals may be associated with a clinical trial of the at least one therapeutic. Further, the processing device 704 may be further configured for analyzing each of the two or more output data based on a predefined therapeutic response. Further, the predefined therapeutic response influences an outcome of the clinical trial. Further, the analyzing of each of the two or more output data may be based on the AI model. Further, the processing device 704 may be further configured for determining two or more targeted individuals from the two or more individuals based on the analyzing of each of the two or more output data. Further, the processing device 704 may be further configured for generating a report data based on the determining of the two or more targeted individuals. Further, the report data corresponds to a report associated with the two or more target individuals. Further, the communication device 702 may be further configured for transmitting the report data to the at least one device.

Further, in some embodiments, the analyzing of each of the two or more output data may include assigning a value to each of the two or more individuals based on the two or more output data. Further, the value corresponds to a coordinate of a multi-dimensional space. Further, the analyzing of each of the two or more output data may include representing each of the two or more individuals as the value in the multi-dimensional space based on the assigning of the value. Further, the determining of the two or more targeted individuals may be based on a proximity between two or more values associated with the two or more individuals in the multi-dimensional space.

In some embodiments, the two or more targeted individuals includes one or more of a placebo responder and a non-placebo responder. Further, the predefined therapeutic response includes one or more of a placebo response and a non-placebo response. Further, the placebo responder experiences a placebo-recovery from the at least one brain disorder. Further, the placebo-recovery may be associated with a placebo effect. Further, the non-placebo responder experiences a therapeutic-recovery from the at least one brain disorder. Further, the therapeutic-recovery may be associated with an active treatment of the at least one therapeutic. Further, the report includes one or more of a placebo responder report and a non-placebo responder report.

In some embodiments, the analyzing of the brain-wave data includes identifying a biological-characteristic of the individual. Further, the electrical activity of the brain may be based on the biological-characteristic. Further, the at least one brain disorder may be associated with the biological-characteristic. Further, the biomarker may be associated with the biological-characteristic.

In some embodiments, the brain-wave dataset includes each of a target-labelled brain-wave data and a target-unlabeled brain-wave data. Further, each of the target-labelled brain-wave data and the target-unlabeled brain-wave data may be recorded from two or more drug-trail participants. Further, the two or more drug-trail participants may be associated with a clinical trial of the at least one therapeutic comprising one or more of a placebo treatment and an active treatment. Further, the target-labelled brain-wave data includes an indicator indicating the at least one therapeutic associated with each of the two or more drug-trail participants. Further, the target-unlabeled brain-wave data lacks the indicator.

In some embodiments, the AI model includes two or more AI models comprising each of the first AI model, a second AI model, a third AI model, and a fourth AI model. Further, the first AI model may be configured to be trained on a target-unlabeled brain-wave data based on a self-supervised learning technique. Further, the brain-wave dataset includes the target-unlabeled brain-wave data. Further, the second AI model may be configured for clustering two or more individuals in a multi-dimensional space. Further, the two or more individuals may be associated with the target-unlabeled brain-wave data.

In some embodiments, the second AI model may be further configured for predicting a biological characteristic associated with each of the two or more individuals. Further, the third AI model may be configured for predicting a response of the two or more individuals to a therapy. Further, the fourth AI model may be configured for predicting the therapeutic response of the two or more individuals to the at least one therapeutic. Further, the fourth AI model may be configured to be trained using a supervised learning.

In some embodiments, the AI model may be based on one or more of a deep convolutional neural network and a generative adversarial network.

In some embodiments, the analyzing of the brain-wave data includes identifying a biomarker in the graphical representation. Further, the biomarker corresponds to an indicator indicating a biological variable associated with the at least one brain disorder. Further, the AI model may be configured to obtain a knowledge of the biomarker associated with the at least one brain disorder based on the brain-wave dataset.

In some embodiments, the biomarker corresponds to a biomarker-graphical representation of a biomarker-electrical activity of the brain. Further, the biomarker-electrical activity corresponds to the at least one brain disorder.

In some embodiments, the analyzing of the brain-wave data includes identifying therapeutic response based on each of the graphical representation and the biomarker.

In some embodiments, the electrical activity may be associated with an electrical signal. Further, the graphical representation corresponds to the electrical signal. Further, the electrical signal may be characterized by an electrical signal characteristic indicating a biological variable associated with the at least one brain disorder.

In some embodiments, the electrical signal characteristic includes an electrical signal frequency.

In some embodiments, the pre-processing of the brain-wave data may be based on a noise filter. Further, the noise-wave data corresponds to a noise electrical signal characterized by a noise frequency. Further, the pre-processing may be further based on each of the noise frequency and a cut-off frequency of the filter.

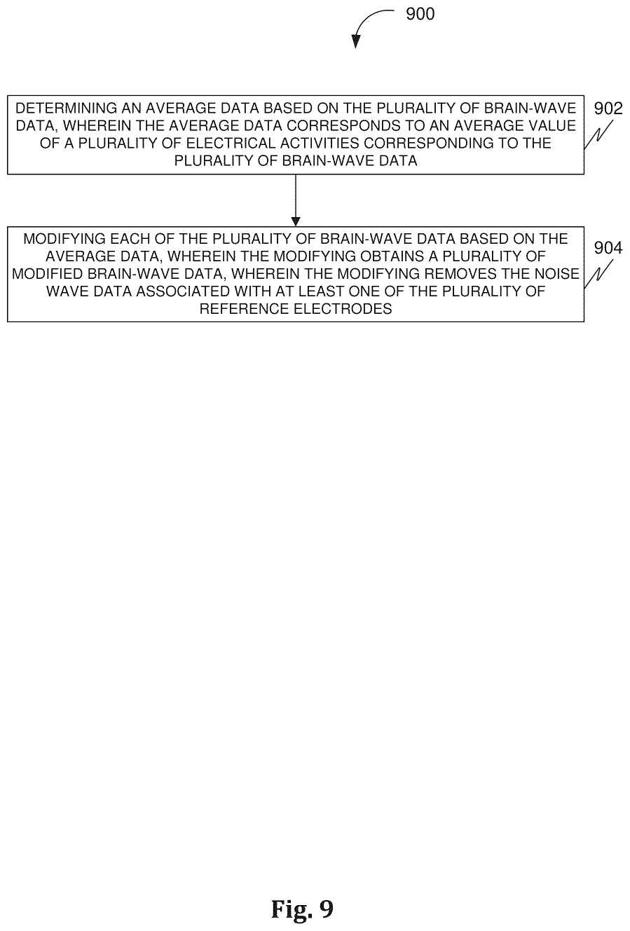

FIG. 9 illustrates a flowchart of a method 900 of EEG-based biomarker discovery and commercialization for personalized diagnosis and treatment of brain disorders including modifying each of the plurality of brain-wave data, in accordance with some embodiments.

Further, in some embodiments, the brain-wave data may be recorded using the diagnostic device 802 may include two or more electrodes. Further, the brain-wave data may include two or more brain-wave data corresponding to the two or more electrodes based on two or more reference electrodes. Further, the individual may be associated with the two or more electrodes. Further, the pre-processing of the brain-wave data may include a step 902 of determining an average data based on the two or more brain-wave data. Further, the average data corresponds to an average value of two or more electrical activities corresponding to the two or more brain-wave data. Further, the brain-wave data may include two or more brain-wave data corresponding to the two or more electrodes based on two or more reference electrodes. Further, the pre-processing of the brain-wave data may include a step 904 of modifying each of the two or more brain-wave data based on the average data. Further, the modifying obtains two or more modified brain-wave data. Further, the modifying removes the noise wave data associated with one or more of the two or more reference electrodes.

In some embodiments, each of the two or more modified brain-wave data may be associated with a common reference.

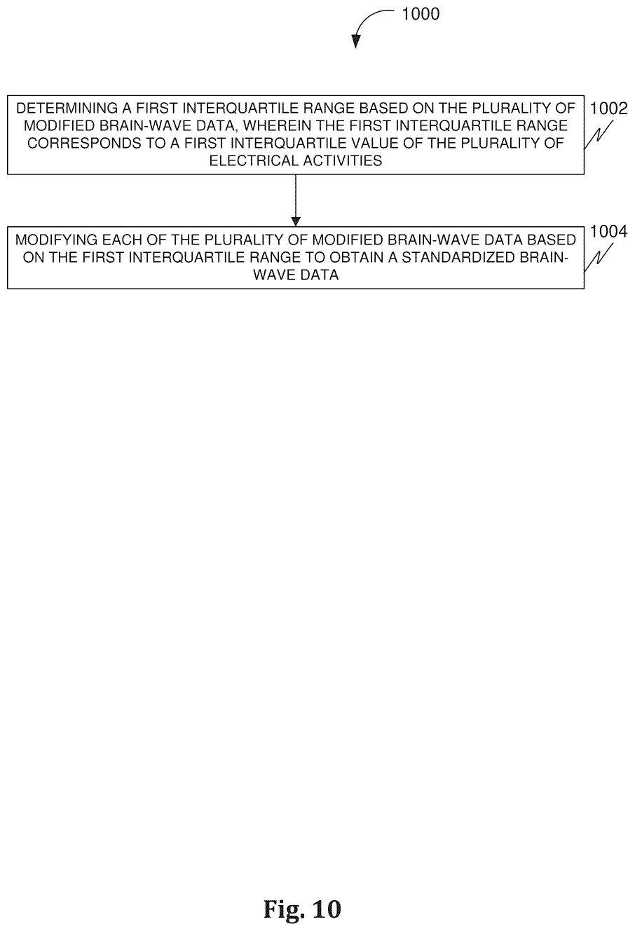

FIG. 10 illustrates a flowchart of a method 1000 of EEG-based biomarker discovery and commercialization for personalized diagnosis and treatment of brain disorders including modifying each of the plurality of modified brain-wave data, in accordance with some embodiments.

Further, in some embodiments, the pre-processing further may include a step 1002 of determining a first interquartile range based on the two or more modified brain-wave data. Further, the first interquartile range corresponds to a first interquartile value of the two or more electrical activities. Further, the pre-processing further may include a step 1004 of modifying each of the two or more modified brain-wave data based on the first interquartile range to obtain a standardized brain-wave data.

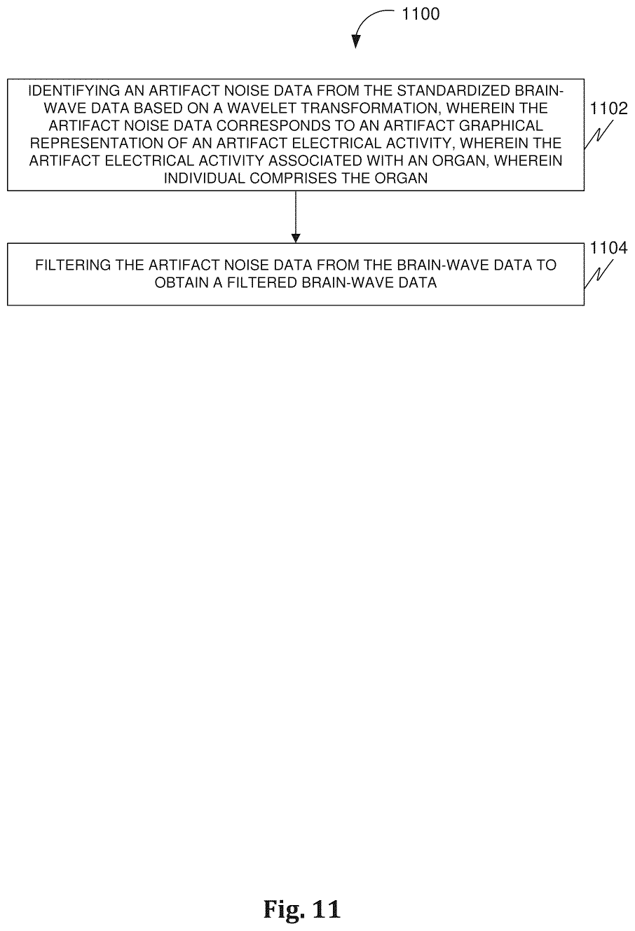

FIG. 11 illustrates a flowchart of a method 1100 of EEG-based biomarker discovery and commercialization for personalized diagnosis and treatment of brain disorders including filtering the artifact noise data from the brain-wave data to obtain a filtered brain-wave data, in accordance with some embodiments.

Further, in some embodiments, the pre-processing further may include a step 1102 of identifying an artifact noise data from the standardized brain-wave data based on a wavelet transformation. Further, the artifact noise data corresponds to an artifact graphical representation of an artifact electrical activity. Further, the artifact electrical activity associated with an organ. Further, individual includes the organ. Further, the pre-processing further may include a step 1104 of filtering the artifact noise data from the brain-wave data to obtain a filtered brain-wave data.

In some embodiments, the organ includes one or more of an eye, a heart and a muscle. Further, the artifact electrical activity of the eye, the heart, and the muscle may be based on a movement of the eye, a beating of the heat, and a muscle activity respectively.

In some embodiments, the noise-wave data may be based on an interference of an external electrical signal associated with a power supply. Further, the brain-wave data may be recorded using the diagnostic device 802. Further, the diagnostic device 802 may be associated an environment associated with the power supply.

In some embodiments, the external electrical signal may be characterized by an electrical signal frequency. Further, the electrical signal frequency ranges from 50 Hz to 60 Hz.

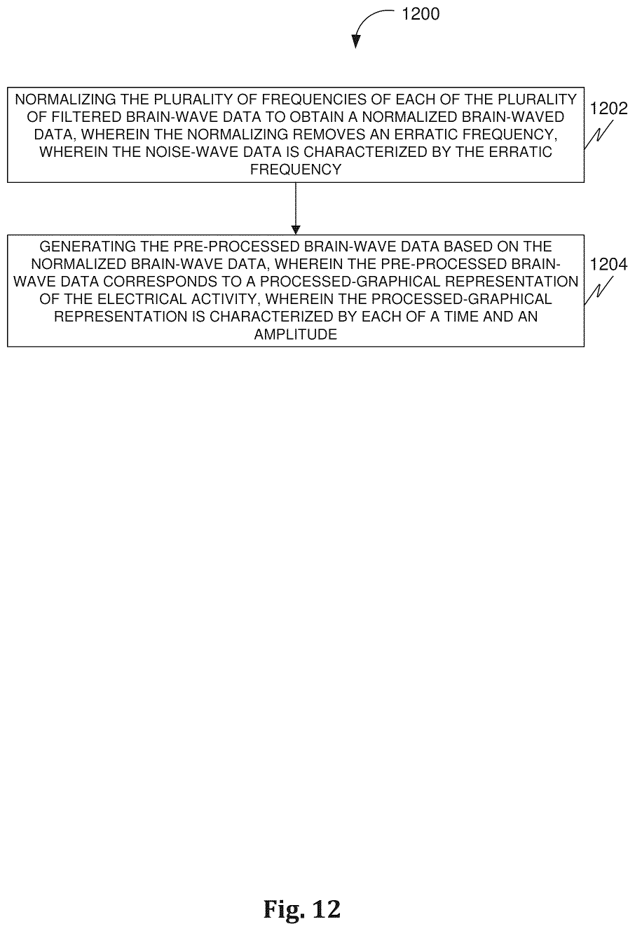

FIG. 12 illustrates a flowchart of a method 1200 of EEG-based biomarker discovery and commercialization for personalized diagnosis and treatment of brain disorders including generating the pre-processed brain-wave data, in accordance with some embodiments.

Further, in some embodiments, the filtered brain-wave data may include two or more filtered brain-wave data corresponding to the two or more electrodes. Further, the two or more filtered brain-wave data may be characterized by two or more frequencies. Further, the pre-processing further may include a step 1202 of normalizing the two or more frequencies of each of the two or more filtered brain-wave data to obtain a normalized brain-waved data. Further, the normalizing removes an erratic frequency. Further, the noise-wave data may be characterized by the erratic frequency. Further, the filtered brain-wave data may include two or more filtered brain-wave data corresponding to the two or more electrodes. Further, the pre-processing further may include a step 1204 of generating the pre-processed brain-wave data based on the normalized brain-wave data. Further, the pre-processed brain-wave data corresponds to a processed-graphical representation of the electrical activity. Further, the processed-graphical representation may be characterized by each of a time and an amplitude.

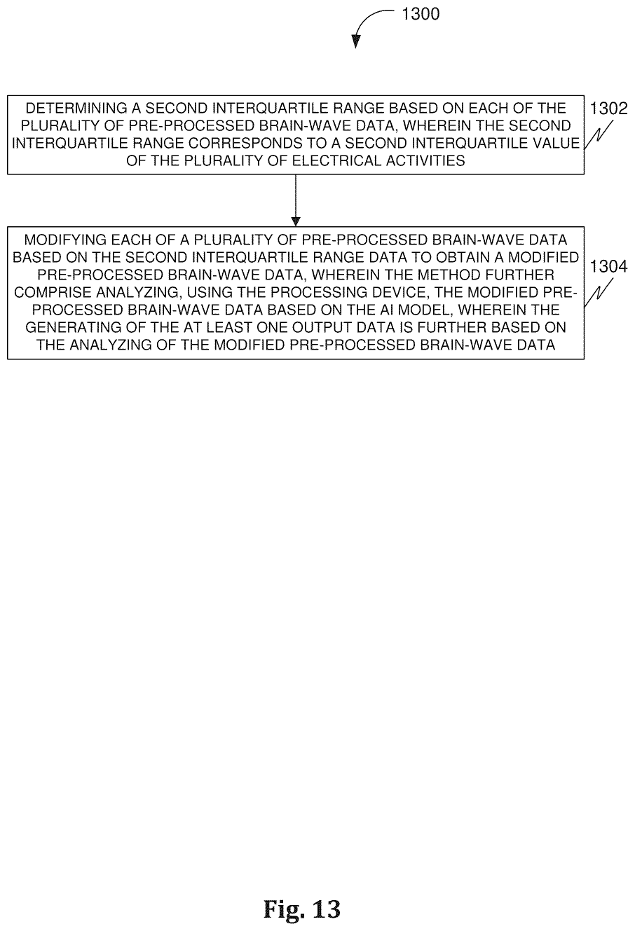

FIG. 13 illustrates a flowchart of a method 1300 of EEG-based biomarker discovery and commercialization for personalized diagnosis and treatment of brain disorders including modifying each of a plurality of pre-processed brain-wave data, in accordance with some embodiments.

Further, in some embodiments, the pre-processed brain-wave data may include two or more pre-processed brain-wave data. Further, the pre-processing further may include a step 1302 of determining a second interquartile range based on each of the two or more pre-processed brain-wave data. Further, the second interquartile range corresponds to a second interquartile value of the two or more electrical activities. Further, the pre-processing further may include a step 1304 of modifying each of two or more pre-processed brain-wave data based on the second interquartile range data to obtain a modified pre-processed brain-wave data. Further, the method 1300 further include analyzing, using the processing device 704, the modified pre-processed brain-wave data based on the AI model. Further, the generating of the at least one output data may be further based on the analyzing of the modified pre-processed brain-wave data.

In some embodiments, the brain-wave data may be generated using the diagnostic device 802 which may be configured for recording the electrical activity. Further, the diagnostic device 802 includes two or more components. Further, the individual includes two or more organs. Further, the recording may be using each of the two or more components and the two or more organs. Further, the recording may be based on a characteristic of one or more of the two or more organs and the two or more components.

In some embodiments, the two or more organs includes one or more of a skull and a skin. Further, the two or more components includes one or more of an electrode, a wire, a preamplifier, and an electrode gel.

In some embodiments, the characteristic includes one or more of a structure of the skull, a property of skin, a quality of the electrode gel, a material of the electrode, and a quality of the electrode.

In some embodiments, the recording may be based on a density of an interaction between the electrode and the skin.

In some embodiments, the noise filter includes one or more of a band-pass filter and a notch filter.

In some embodiments, the pre-processing may be further based on a median filter.

In some embodiments, the modifying based on the second interquartile range data removes an amplitude outlier. Further, the pre-processed brain-wave data includes the amplitude outlier.

In some embodiments, the brain-wave data includes a first-brain wave data and a second brain wave data. Further, the first-brain wave data and the second brain wave data corresponds to a first diagnostic tool and a second diagnostic tool respectively. Further, the first-brain wave data and the second brain wave data may be characterized by a first format and a second format respectively. Further, the first format may be different form the second format.

In some embodiments, the pre-processing facilitates normalizing each of the first format and the second format.

In some embodiments, the AI model may be configured to pre-process the brain-wave data characterized by two or more formats.

In some embodiments, the AI model includes a ML model.

In some embodiments, the multi-dimensional space includes a 128-dimension space.

In some embodiments, the graphical representation of each of the two or more targeted individuals indicates a unique pattern. Further, the unique pattern corresponds to an EEG fingerprint.

In some embodiments, the graphical representation of each of the two or more targeted individuals indicates a dynamic pattern. Further, the dynamic pattern indicates one or more of two or more stages of the at least one brain disorder.

In some embodiments, the electrical activity of the brain may be based on one or more of the at least one brain disorder and the at least one therapeutic.

In some embodiments, the two or more targeted individuals includes two or more healthy individuals.

In some embodiments, the two or more targeted individuals includes two or more diseased individuals associated with one or more of two or more stages of the at least one brain disorder. Further, the predefined therapeutic response corresponds to one or more of two or more stages of the at least one brain disorder.

In some embodiments, the at least one brain disorder includes a depression. Further, the at least one therapeutic includes an antidepressant treatment.

In some embodiments, the antidepressant treatment may be based on an antidepressant drug. Further, the individual consumes the antidepressant drug. Further, the electrical activity may be based on the antidepressant drug.

In some embodiments, the analyzing each of the two or more output data includes analyzing a neural circuit characteristic of the two or more individuals. Further, the analyzing predicts a change associated with the two or more individuals in response to the at least one therapeutic.

In some embodiments, the brain-wave dataset includes two or more placebo brain-wave data corresponding two or more placebo-graphical representations. Further, the two or more placebo-graphical representations indicates a placebo-electrical activity associated with a placebo-responder.

In some embodiments, the AI model may be associated with a transfer learning technology. Further, the AI model may be trained on a first task to obtain a first knowledge. Further, the AI model may be configured to perform a second task on the first knowledge. Further, the first task may be different from the second task.

In some embodiments, the predefined therapeutic response may be associated with one or more of a rostral theta current density, an alpha-band connectivity, and a gamma-band connectivity.

In some embodiments, the gamma-band connectivity corresponds to a parietal region of the brain.

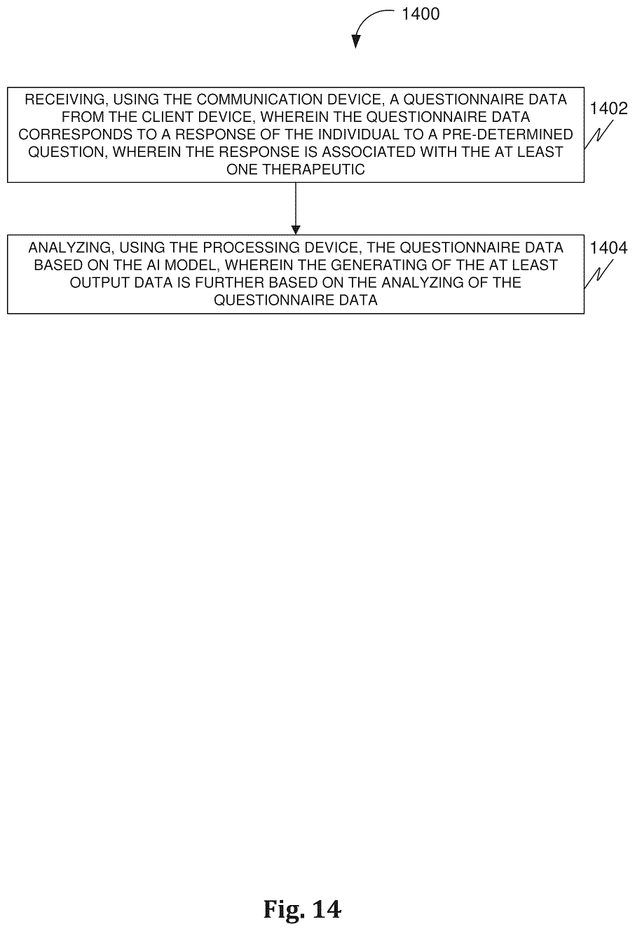

FIG. 14 illustrates a flowchart of a method 1400 of EEG-based biomarker discovery and commercialization for personalized diagnosis and treatment of brain disorders including analyzing, using the processing device 704, the questionnaire data, in accordance with some embodiments.

Further, in some embodiments, the method 1400 further may include a step 1402 of receiving, using the communication device 702, a questionnaire data from the at least one device. Further, the questionnaire data corresponds to a response of the individual to a pre-determined question. Further, the response may be associated with the at least one therapeutic. Further, in some embodiments, the method 1400 further may include a step 1404 of analyzing, using the processing device 704, the questionnaire data based on the AI model. Further, the generating of the at least one output data may be further based on the analyzing of the questionnaire data.

In some embodiments, the method 500 may further include generating, using the processing device 704, a score data based on the analyzing of each of the two or more output data. Further, the score data corresponds to a score of each of the two or more individuals in relation to each of the two or more output data and the predefined therapeutic response. Further, the determining of the two or more targeted individuals may be based on the score.

In some embodiments, the two or more participants includes one or more of a placebo responder and a non-placebo responder. Further, the placebo responder may be associated with a first score. Further, the non-placebo responder may be associated with a second score. Further, the first score may be greater than the second score. Further, the predefined therapeutic response corresponds to the placebo responder.

In some embodiments, the clinical trial includes one or more of a phase-two trail and a phase-three trail.

In some embodiments, the two or more brain-wave data corresponds to one or more of the phase-two trail and the phase-three trail.

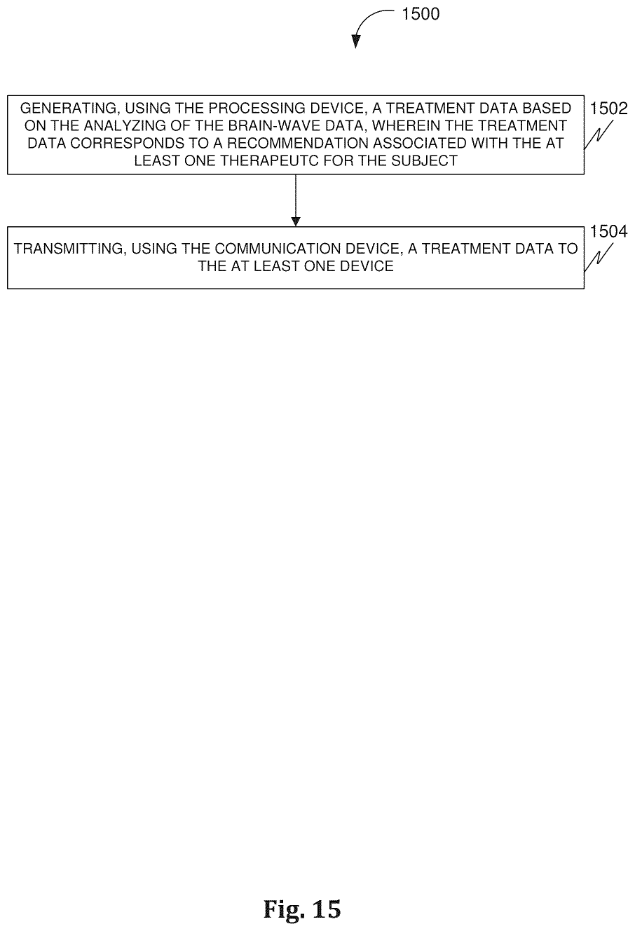

FIG. 15 illustrates a flowchart of a method 1500 of EEG-based biomarker discovery and commercialization for personalized diagnosis and treatment of brain disorders including generating, using the processing device 704, a treatment data, in accordance with some embodiments.

Further, in some embodiments, the method 1500 further may include a step 1502 of generating, using the processing device 704, a treatment data based on the analyzing of the brain-wave data. Further, the treatment data corresponds to a recommendation associated with the at least one therapeutic for the individual. Further, in some embodiments, the method 1500 further may include a step 1504 of transmitting, using the communication device 702, a treatment data to the at least one device 800.

In some embodiments, the analyzing of the brain-wave data includes identifying a biological-characteristic of the individual based on the electrical activity. Further, the electrical activity further based on a biological-characteristic. Further, the at least one brain disorder may be associated with the biological-characteristic. Further, the biomarker may be associated with the biological-characteristic. Further, the generating of the treatment data may be based on the biological-characteristic.

In some embodiments, the biological-characteristic includes one or more of a gender and an age.

In some embodiments, the at least one brain disorder may be associated with a brain-sex phenotype.

In some embodiments, the recommendation includes a biological-characteristic based recommendation.

In some embodiments, the analyzing of the brain-wave data further includes identifying a type of the at least one brain disorder based on the biological-characteristic.

In some embodiments, the AI model may be validated based on two or more validation methods. Further, the two or more validation methods includes each of an internal validation method and an external validation method.

In some embodiments, the internal validation method may be based on two or more training datasets comprising the brain-wave dataset.

In some embodiments, the external validation method may be based on two or more external datasets. Further, the two or more training datasets may be different from the two or more external datasets.

In some embodiments, the two or more targeted individuals includes a suitable candidate for the clinical trial. Further, the brain-wave data corresponds to a phase-two clinical trial. Further, the report data corresponds to a selection of the two or more targeted individuals for a phase-three clinical trial.

In some embodiments, the report may include one or more of a study-fit score of the individual, an expected response, a recommendation, and a subject profile.

In some embodiments, the two or more targeted individuals includes a psychiatric therapy responder. Further, the psychiatric therapy responder experiences a recovery from a psychiatric disorder in response to the psychiatric therapy.

In some embodiments, the pre-determined question includes a depression question.

In some embodiments, the depression question corresponds to one or more of Hamilton depression rating scale-17, montgomery-Asberg depression rating scale, patient health questionnaire-9, work and social adjustment scale, pittsburgh sleep quality index, anxiety and depression questionnaire-7, screening for healthy, active participation support, and quick inventory of depressive symptomatology.

In some embodiments, the individual may be associated with one or more of two or more clinical trials. Further, the individual may include a drug trail participant. Further, the at least one therapeutic may include the at least one drug.

In some embodiments, individual may be associated with a task. Further, the electrical activity of the brain may be based on the task.

In some embodiments, the pre-processing may be based on a statistical characteristic of the brain-wave data.

In some embodiments, the clustering of the two or more individuals may be based on the self-supervised learning of the first AI model.

In some embodiments, the first AI model includes a masked variational autoencoder.

In some embodiments, the second AI model includes a conditional variational autoencoder.

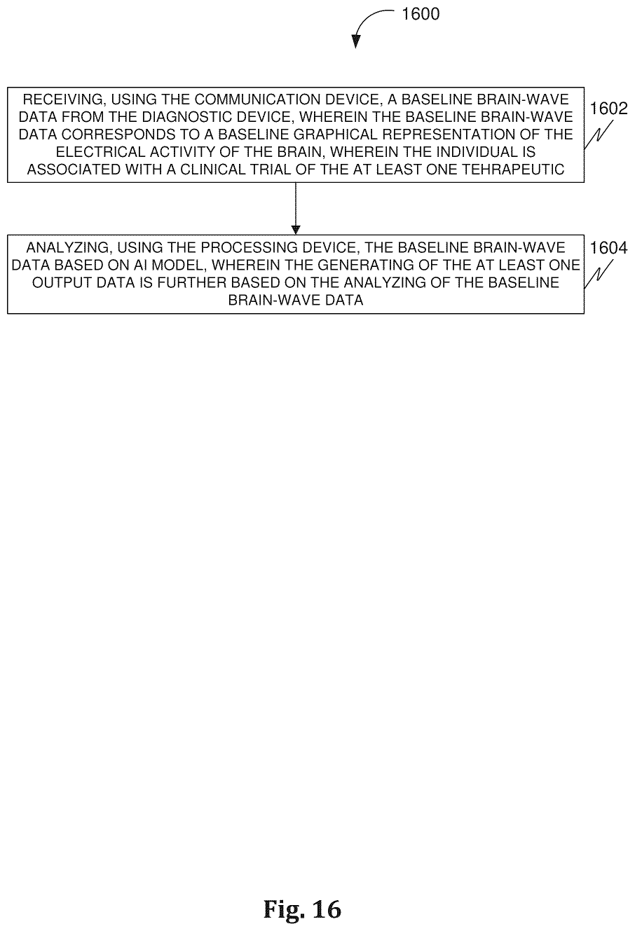

FIG. 16 illustrates a flowchart of a method 1600 of EEG-based biomarker discovery and commercialization for personalized diagnosis and treatment of brain disorders including analyzing, using the processing device 704, the baseline brain-wave data, in accordance with some embodiments.

Further, in some embodiments, the method 1600 further may include a step 1602 of receiving, using the communication device 702, a baseline brain-wave data from the diagnostic device 802. Further, the baseline brain-wave data corresponds to a baseline graphical representation of the electrical activity of the brain. Further, the individual may be associated with a clinical trial of the at least one therapeutic. Further, in some embodiments, the method 1600 further may include a step 1604 of analyzing, using the processing device 704, the baseline brain-wave data based on AI model. Further, the generating of the at least one output data may be further based on the analyzing of the baseline brain-wave data.

In some embodiments, the brain-wave data include a follow-up brain-wave data. Further, the baseline brain-wave data may be recorded at a first time instance. Further, the follow-up brain-wave data may be recorded at a second time instance. Further, the second time instance occurs after the first time instance. Further, the first time instance and the second time instance may be associated with a first stage of a clinical trial and a second of a clinical trial respectively. Further, the first stage may be different from the second stage.

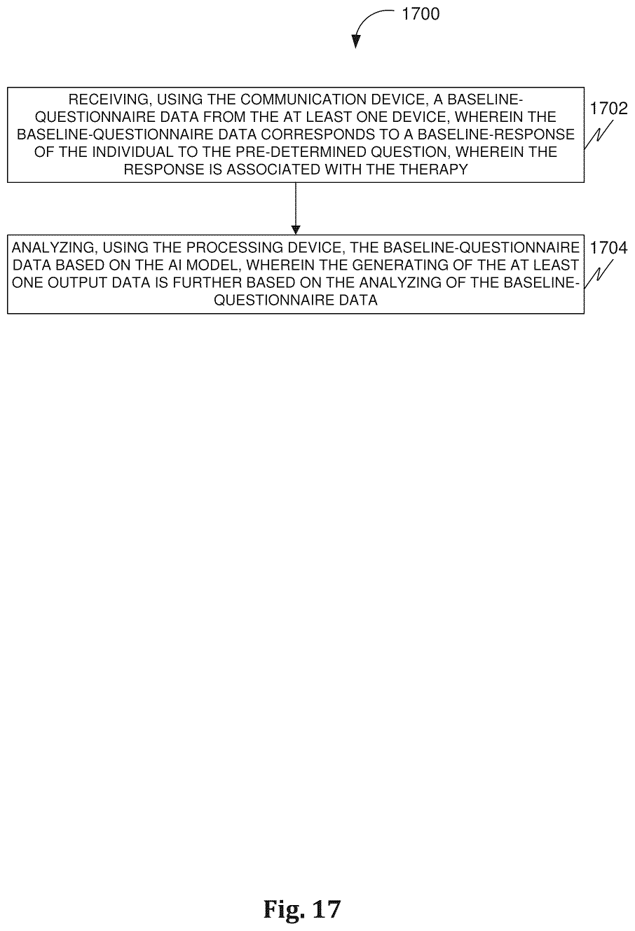

FIG. 17 illustrates a flowchart of a method 1700 of EEG-based biomarker discovery and commercialization for personalized diagnosis and treatment of brain disorders including analyzing, using the processing device 704, the baseline-questionnaire data, in accordance with some embodiments.

Further, in some embodiments, the method 1700, further may include a step 1702 of receiving, using the communication device 702, a baseline-questionnaire data from the at least one device. Further, the baseline-questionnaire data corresponds to a baseline-response of the individual to the pre-determined question. Further, the response may be associated with the at least one therapeutic. Further, in some embodiments, the method 1700, further may include a step 1704 of analyzing, using the processing device 704, the baseline-questionnaire data based on the AI model. Further, the generating of the at least one output data may be further based on the analyzing of the baseline-questionnaire data.

In some embodiments, the questionnaire data follow-up questionnaire data. Further, the baseline-questionnaire data may be recorded at the first time instance. Further, the follow-up questionnaire data may be recorded at the second time instance.

In some embodiments, the AI model may be trained on each of the baseline brain-wave data, the follow-up brain-wave data, the baseline-questionnaire data, and the follow-up questionnaire data.

In some embodiments, the pre-processed brain-wave data includes a denoised EEG data.

In some embodiments, the representing of the plurality of the individuals includes clustering of the two or more brain-wave data in the multi-dimensional space.

In some embodiments, the AI may be trained to identify a biomarker associated with each of the placebo treatment and the active treatment.

In some embodiments, the AI model may be configured to adapt to two or more diseases associated with two or more therapies.

In some embodiments, the pre-processing enhances a quality of the EEG data.

In some embodiments, the brain-wave data includes two or more brain-wave data. Further, the two or more brain-wave data may be associated with two or more formats. Further, the pre-processing includes one or more of a data normalization, a noise removal and equalization. Further, the pre-processed brain-wave data includes a combined-brain wave data.

In some embodiments, each the two or more brain-wave data may be associated with a distinct source.

In some embodiments, the pre-processing reduces the data leakage associated with the noise-wave data.

In some embodiments, the AI model may be further configured to identify a data leakage associated with one or more of an equipment-noise and a site-noise.

In some embodiments, the AI model includes a EEG data whitening model comprising a data preprocessing model, a data cleaning model, etc.

In some embodiments, the AI model may be configured for pre-processing the EEG data, labelling the EGG data, and differentiating an EEG signal from a noise signal.

In some embodiments, the brain-wave dataset includes each of a publicly available EEG data and a non-publicly available EEG data.

In some embodiments, the brain-wave dataset may be associated with two or more electrode placements. Further, the two or more electrode placements includes at 10/5 electrode placement and a geodesic sensor placement.

In some embodiments, the brain-wave dataset may be associated with two or more states. Further, the two or more states includes one or more of a resting state and a task state. Further, the brain-wave dataset includes one or more of a rest-state data and a task-state data.

In some embodiments, the resting state includes one or more of an open-eye state and a closed-eye state.

In some embodiments, the individual associated with the task state performs a twenty different task.

In some embodiments, the brain-wave dataset includes a metadata corresponding to an attribute of one or more of the individual and a recording session. Further, the brain-wave dataset may be recorded at the recording session.

In some embodiments, the analyzing of the brain-wave data includes a calculating a pre-selected quantitative characteristic based on the brain-wave data associated with the at least one therapeutic.

In some embodiments, the pre-selected quantitative characteristic includes a pre-selected quantitative EEG characteristic.

In some embodiments, the AI model may be configured to calculate four thousand pre-selected quantitative EEG characteristics.

In some embodiments, the AI model includes a placebo non-responder AL model. Further, the brain-wave dataset includes a EEG placebo dataset.

In some embodiments, the EEG placebo dataset may be associated with European multicenter bronchiectasis audit and research collaboration (EMBARC) study.

In some embodiments, the score data may be associated in segmenting the two or more individuals.

In some embodiments, the score includes a prognostic score of change in the at least one brain disorder. Further, the change may be based on one or more of a placebo treatment and an active treatment. Further, the at least one therapeutic includes one or more of the placebo treatment and the active treatment.

In some embodiments, the score includes a binary value comprising one or more of a zero and one, may. Further, the zero associated with a non-responder to one or more of a placebo treatment and an active treatment. Further, the at least one therapeutic includes one or more of the placebo treatment and the active treatment. Further, the one associated with a responder to one or more of the placebo treatment and the active treatment.

In some embodiments, the score corresponds to a probability measure ranging from one to zero. Further, the probability measure indicates a probability of responds to the at least one therapeutic.

In some embodiments, the at least one output data associated with determining an effectiveness of the at least one therapeutic.

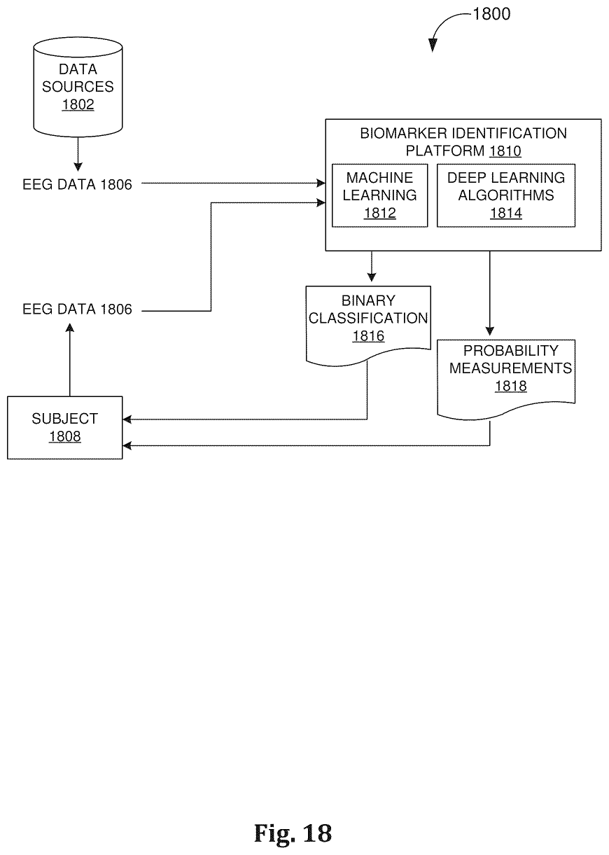

FIG. 18 illustrates a system 1800 for identifying EEG-based pharmacologically significant biomarker, in accordance with some embodiments.

In some embodiments, the system 1800 and method for identifying EEG-based pharmacologically significant biomarkers may function as a biomarker identification platform 1810 to identify novel biomarkers utilizing a plurality of deep learning models. The system and method may be implemented in both clinical and pre-clinical settings and may be utilized to generate binary classifications 1816, probability measurements 1818, or a prognostic score.

In some embodiments, the system 1800 and method utilize an EEG reading collected from a subject 1808 as input. The EEG data 1806 may include the EEG reading and include clinical data, demographic data, questionnaire data, EMR/EHR, etc. The EEG data 1806 is then processed utilizing AI/machine learning 1812/deep learning techniques 1814 and then utilized to predict a subject's response to a specific treatment or placebo, whether during a clinical trial or in a clinical setting. Further, the system 1800 may utilize the EEG data 1806 from data sources 1802.

The system 1800 and method may be implemented with pharmaceutical treatments administered via different routes (e.g., orally, nasally, intravenously, intramuscular, etc.), as well as TMS/rTMS/dTMS, or placebos. These treatments can be intended for specific psychiatric conditions, such as depression.

In some embodiments, the prediction generated through the system 1800 and method may be output as a binary classification 1816, probability measurement 1818, or a prognostic score. The binary classification 1816 identifies whether the subject 1808 is either a responder (1) or a non-responder (0) to the selected treatment (including placebo). The probability measure 1818 (ranging between 0 and 1, or expressed as a percentage) indicates the subject's likelihood of responding-or not-to the treatment. The prognostic score indicates the score (or percentage change from baseline) the subject 1808 would have if the subject 1808 was in the control group (placebo) or a treatment group.

In some embodiments, the system 1800 and method may be utilized in clinical trials to predict subject's prognostic score. The subject's prognostic score represents the subject's expected score (or the score percentage change from baseline) on a specific assessment scale (e.g., HDRS17, MADRS) at the end of the treatment course, assuming the subject 1808 receives either the treatment or placebo.

The prognostic score may be used as a covariate in the statistical analysis of the clinical trial's results using ANCOVA (analysis of covariance) or MMRM (mixed model for repeated measures) method, thereby enhancing trial efficiency.

In some configurations, EEG data 1806 may be collected either in a resting state, known as “resting state EEG,” or while the subject 1808 performs certain tasks, commonly referred to as ERP (event-related potential). These tasks can be cognitive or others, such as the emotional recognition task, Stroop task, N-back task, Stop-signal task, or probabilistic reward task.

In some embodiments, the system 1800 and method may be utilized in clinical trials as well as in clinical settings. In a clinical trial, the system 1800 and method may be utilized in predicting placebo responders and non-responders from EEG data 1806, thereby enhancing trial efficiency. Additionally, the system 1800 and method may be able to identify treatment responders and non-responders from EEG data 1806. In a clinical setting, the system 1800 and method may serve as a companion diagnostic test to predict a patient's response to a particular treatment, thus assisting healthcare professionals in selecting the most suitable treatment option.

In some embodiments, machine learning models 1812 of the system 1800 and method may be organised as a multilayer training architecture. The first layer is a self-supervised layer, the second layer would be a conditional and masked variational autoencoder, the third layer would be a classification layer, and the fourth layer would be a supervised learning layer. The first layer (self-supervised learning) may include a masked variational autoencoder to take advantage of a large volume of unlabeled EEG records. The second layer (Conditional +masked variational autoencoder) may function as an extension of the first layer and incorporate AI models for subject clustering, sex & age prediction. The third layer may be built on top of the second layer and may operate as a classifier for predicting patient response to any treatment, including placebo. The fourth layer (supervised learning) may be operated as a targeted classifier and may be used to predict patient response to specific treatments.

In some embodiments, the first layer may be implemented using a pretrained foundational transformer model, which is modified to accommodate EEG input data. Subsequently, the second layer could be implemented as a fine-tuned transformer model. During the fine-tuning process, various labelled datasets can be employed, including, but not limited to, the sex and age of subjects.

In some embodiments, an algorithm may be designed to receive either a raw resting-state EEG of the subject 1808, encompassing two sessions (with eyes open and eyes closed), or just one of these sessions, and/or the subject's ERP sessions and/or transformed EEG data, including but not limited to STFT, MEL, superlet and wavelet transformations. The data may be compatible with any of the existing EEG data recording formats, including but not limited to .edf, .eeg, .bdf, etc. Upon analyzing the input data, the algorithm may generate the binary classification 1816, the measure of probability 1818, or a prognostic score. The binary classification 1816 of the subject 1808 may identify if the subject 1808 is a responder or non-responder to treatment (or placebo). Being classified as a responder indicates the algorithm's expectation that the subject 1808 will demonstrate a positive response to the treatment (or placebo) by the end of the treatment course. The subject's prognostic score represents the subject's expected score (or the percentage change from baseline) on a specific assessment scale (e.g., HDRS17, MADRS) at the end of the treatment course, assuming the subject 1808 receives either the treatment or placebo. The subject's probability measurement represents the likelihood of that subject 1808 responding to a specific treatment (or placebo).

In some embodiments, the system 1800 and method may utilis a Deep convolutional neural network (DCNN) transfer learning approach, self-supervised and supervised learning techniques.

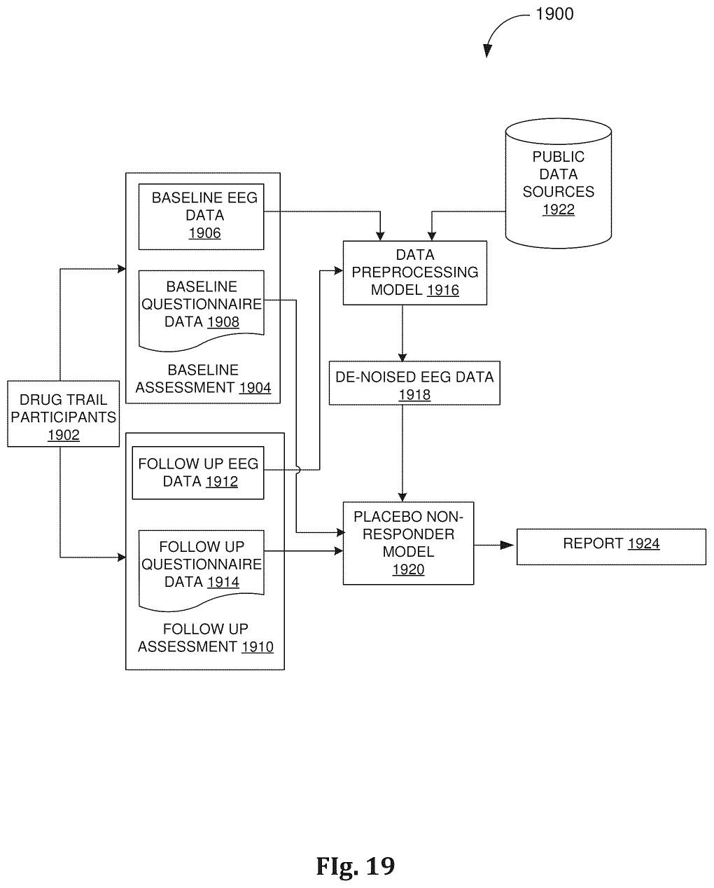

FIG. 19 illustrates a system 1900 for predicting effectiveness of a drug trial on participants, in accordance with some embodiments.

In some embodiments, the system 1900 for predicting effectiveness of a trial drug treatment on participants is provided as a way of screening drug trial participants 1902 for improving the results of the drug trial. In particular, the drug trials for depression and other psychiatric drugs are of particular importance due to various factors associated with psychiatric drug treatments that result in a higher level of failure during pharmaceutical trials. Further, the system 1900 may correlate biometric data collected from the drug trial participants 1902 and taken at various stages of a drug trial, to help improve the determination of the drug's effectiveness. Further, the system 1900 may identify biomarkers in Electroencephalographs (EEG) taken during a drug trial participant's enrollment that assist in screening the drug trial participant 1902 for eligibility in the drug trial.

In some embodiments, the system 1900 for predicting the effectiveness of the drug trial on the drug trial participants 1902 involves collecting publicly available biometric data sets for training of a data preprocessing model 1916. Further, the system 1900 then perform a baseline assessment 1904 of collecting questionnaire data 1908, and Electroencephalographs (EEG) data 1906 of the drug trial participants 1902. The EEG data 1906 is then passed through the data preprocessing model 1916 to de-noise the EEG data to obtain denoised EEG data 1918. The denoised EEG data 1918 is then passed to a treatment (incl. placebo) classification or regression model 1920 for training with the baseline questionnaire data 1908. The system 1900 then perform at least one follow up assessment 1910 after a predetermined number of weeks after the drug trial participants 1902 have been on trialed pharmaceutical, where a follow up questionnaire data 1914 and a follow up EEG data 1912 are collected. The follow up EEG data 1912 is again passed through the data preprocessing model 1916 and passed to the treatment (incl. placebo) classification or regression model 1920 for training with follow up questionnaire data 1914 and the specific pharmaceutical (e.g., trial drug or placebo) being administered. The combination of data sets from the baseline assessment 1904 and follow up assessment 1910 are utilized by the treatment (incl. placebo) classification or regression model 1920 to identify biomarkers from the drug trial participants 1902. Further, the drug trail participants 1902 may have a response to the trial drug, have a response to the placebo, and unlikely have a response to the trial drug in the form of a report 1924. The trained treatment (incl. placebo) non-responder model 1920 may then be utilized to screen additional drug trial participants 1902 for the trial drug.

In some embodiments, the system 1900 may use clustering of EEG data in a multi-dimensional space to train a data clustering model.

In some embodiments, the data preprocessing model 1916 may be utilized in combination with publicly available EEG data from a public data source 1922 and drug trial collected EEG data to denoise the data sets for training a brain sex prediction model that identifies brain sex based biomarkers that could potentially affect the drug trial participant 1902 response to a trial drug or a placebo.

In some embodiments, the system 1900 may utilize a predictive model trained on labelled and non-labelled patient biometric data.

In some embodiments, the system 1900 utilizes a combination of data sources that are collected during a drug trial, which are then provided as training data for machine learning and deep learning models to identify biomarkers of treatment response to the specific treatment (or placebo) that can affect the results of a drug trial. These trained models are then utilized for screening of additional applicants in the same or other drug trials.

In some embodiments, the drug trial participants 1902 may undergo a screening visit that is utilized to qualify the individual into the drug trial. The screening visit is utilized to determine the current condition or baseline of the participant. The screening visit involves at least one of a depression questionnaire and an EEG screening, where the depression questionnaire may score the individual using the Hamilton depression score (HAM-D), MADRS, or other relevant assessment scale.

In some embodiments, the treatment (incl. placebo) response prediction model 1920 may be able to make predictions before randomization or between assessments to determine the responsiveness of the drug trial participants 1902 to the treatment (i.e., trial treatment or placebo) as well as determine a prognostic score.

In some embodiments, the models may be provided with target labelled EEG and non labelled EEG data for training. The target labelled EEG data is collected from the drug trial participants 1902 subjected to at least one of an active treatment and a placebo. Non labelled EEG data is data where the treatment or the results of the treatment (or placebo) are unknown (i.e., trial drug or placebo). By utilizing target labelled EEG data for placebo participants, the models may be trained to identify biomarkers in the drug trial participants 1902 that are placebo responding individuals.

In some embodiments, medical history may be provided to the models to help identify and associate biomarkers with historic data, such that a combination of the drug trial participants 1902 medical history and a biomarker in their EEG may identify if they will be suitable for a novel drug trial.

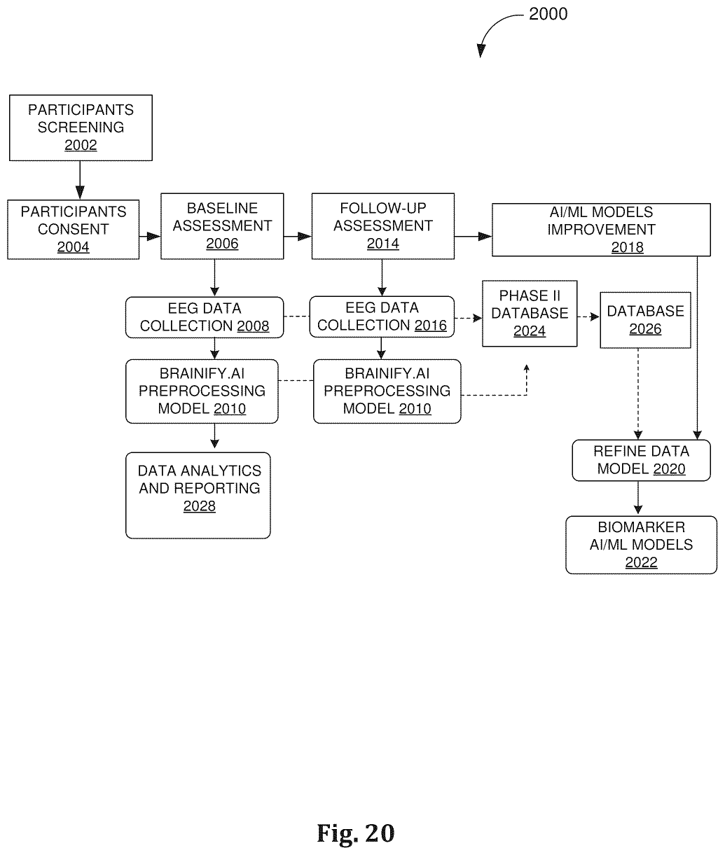

FIG. 20 illustrates a system and method pathway 2000 for phase II trials, in accordance with some embodiments. Further, the system and method pathway 2000 for phase II trials includes participant screening 2002, a participant consent 2004, a baseline assessment 2006, a follow-up assessment 2014, and an AI/ML model improvement 2018. Further, the baseline assessment 2006 may includes each of an EEG data collection 2008 and a preprocessing using a Brainify.AI preprocessing model 2010. Further, the baseline assessment 2006 includes data analytics and reporting 2028. Further, the follow-up assessment 2014 may includes each of an EEG data collection 2016 and a preprocessing using the Brainify.AI preprocessing model 2010. Further, the EEG data collection 2016 and the EEG data collection 2008 is stored in a phase II database 2024 and combined in a Brainify.AI database 2026. Further, the AI/ML model improvement 2018 is based on each of the baseline assessment 2006 and the follow-up assessment 2014. Further, the AI/ML model improvement 2018 may provide a refined data model 2020 and biomarker AI/ML models 2022. Further, the AI/ML model improvement 2018 is using a data from the Brainify.AI database 2026.

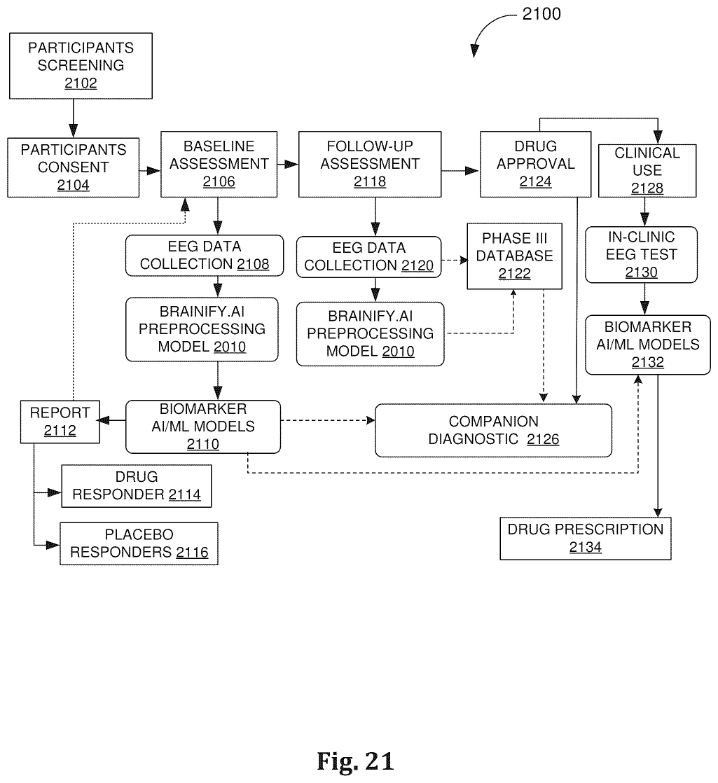

FIG. 21 illustrates a system and method pathway 2100 for phase III trials and FDA regulation, in accordance with an exemplary embodiment. Further, the system and method pathway 2100 for phase III trials includes participant screening 2102, a participant consent 2104, a baseline assessment 2106, a follow-up assessment 2118, a drug approval 2124, and a clinical use 2128. Further, the baseline assessment 2106 includes an EEG data collection 2108, a preprocessing using a Brainify.AI preprocessing model 2010, and identifying a biomarker using a biomarker AI/ML model 2110. Further, the biomarker AI/ML model 2110 may provide a report 2112 that corresponds to one or more of a drug responder 2114 and a placebo responder 2116. Further, the follow-up assessment 2118 includes an EEG data collection 2120 and a preprocessing using the Brainify.AI preprocessing model 2010. Further, data from each of the EEG data collection 2120 and the EEG data collection 2108 is stored in a phase II database 2122. Further, the drug approval 2124 is based on a companion diagnostic 2126, which may be based on the biomarker AI/ML model 2110. Further, the clinical use 2128 includes an in-clinic EEG test 2130. Further, the clinical use 2128 is based on the drug approval 2124. Further, data from the in-clinic EEG test is analyzed by a biomarker AI/ML model 2132 to provide a drug prescription 2134.

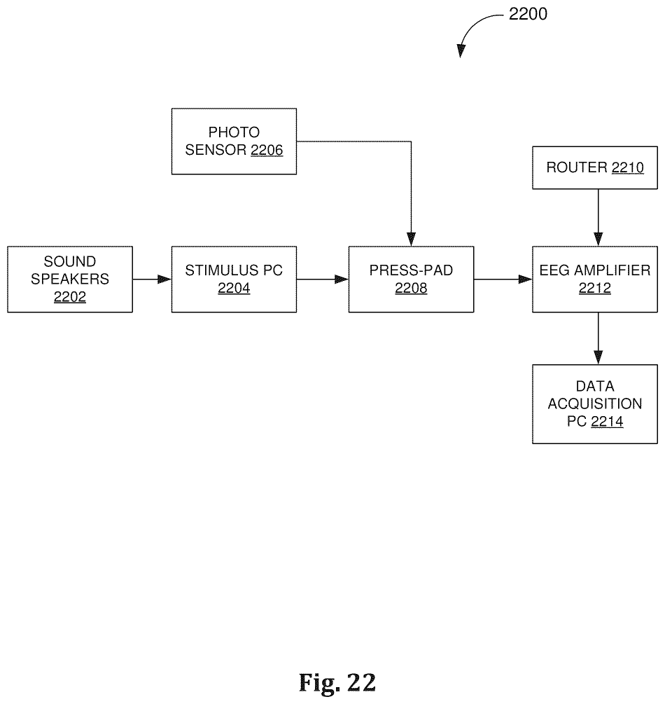

FIG. 22 illustrates an equipment and tools 2200 utilized during data collection by the system and method, in accordance with an exemplary embodiment. Further, the equipment and tools 2200 may include one or more of a sound speaker 2202, a photo sensor 2206, a stimulus PC 2204, a press-pad 2208, an EEG amplifier 2212, a router 2210, and a data acquisition PC 2214.

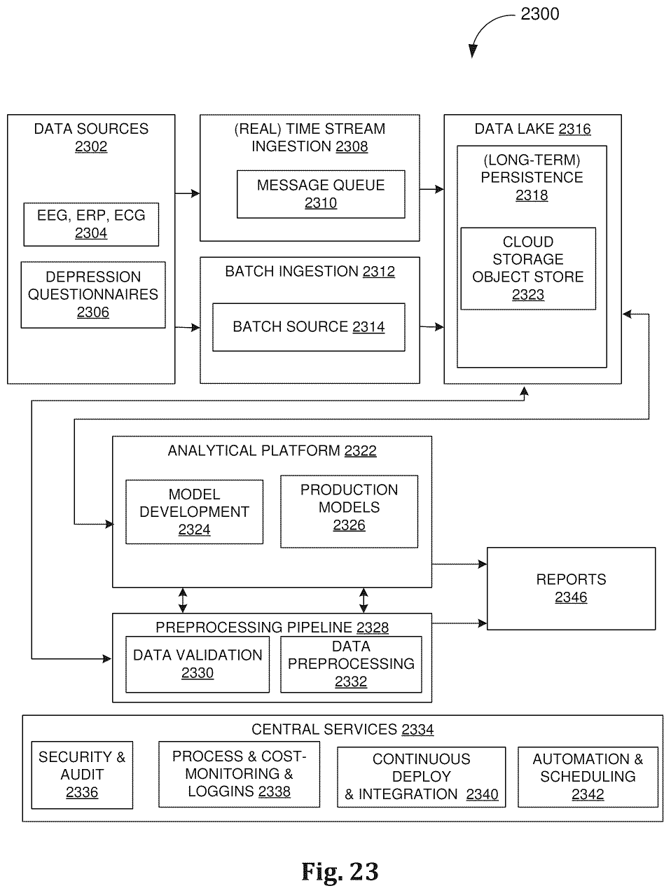

FIG. 23 illustrates a technical architecture 2300 of the system and method, in accordance with some embodiments. Further, the technical architecture 2300 may include one or more of data sources 2302, a real time stream ingestion 2308, a batch ingestion 2312, a data lake 2316, an analytical platform 2322, and a preprocessing pipeline 2328. Further, the analytical platform 2322 and the preprocessing pipeline 2328 may provide reports 2346. Further, the system and method may facilitate central services 2334 including one or more of a security and audit 2336, a process, cost-monitoring and logging 2338, a continuous deployment and integration 2340, and automation scheduling 2342. Further, the analytical platform 2322 may facilitate a model development 2324 that results in a production model 2326. Further, the preprocessing pipeline 2328 may provide data validation 2330 and a data pre-processing 2332. Further, the real time stream ingestion 2308 and the batch ingestion 2312 may include a message queue 2310 and a batch source 2314 respectively. Further, the data lake 2316 may include cloud storage object store 2320 with long term persistence 2318. Further, the data sources 2302 may include one or more of EEG, ERP and ECG 2304. Further, the data sources 2302 may include depression questionnaires 2306.

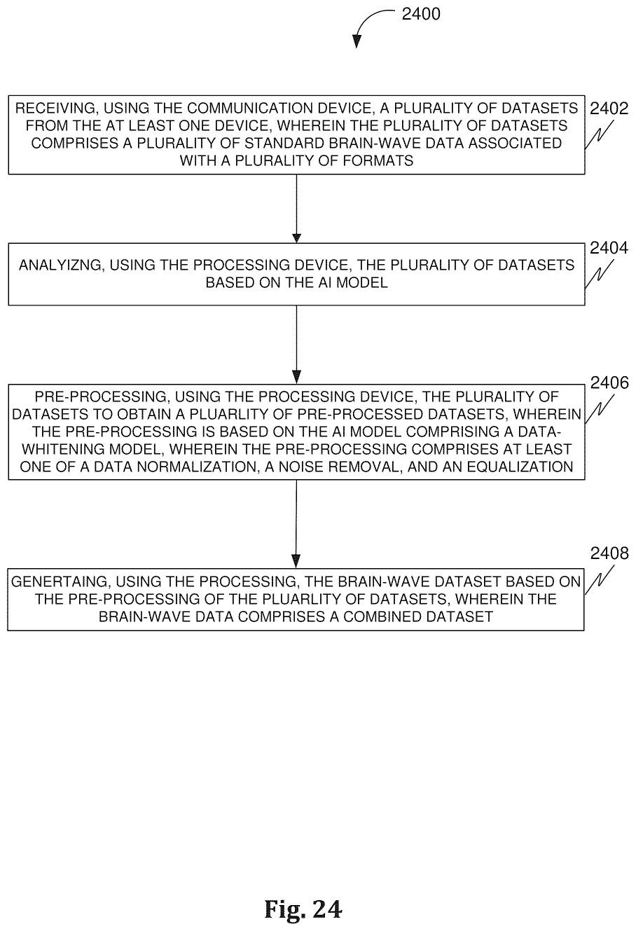

FIG. 24 illustrates a flowchart of a method 2400 of EEG-based biomarker discovery and commercialization for personalized diagnosis and treatment of brain disorders including generating, using the processing device 704, a brain-wave dataset using a data-whitening model, in accordance with some embodiments.

Further, in some embodiments, the method 2400 may include a step 2402 of receiving, using the communication device 702, two or more datasets from the at least one. Further, the two or more datasets comprises a two or more standard brain-wave data associated with two or more formats. Further, the 2400 may include a step 2404 of analyzing, using the processing device 704, the two or more datasets based on the AI model. Further, the method 2400 may include a step 2406 of pre-processing, using the processing device 704, the two or more datasets to obtain the two or more pre-processed datasets. Further, the pre-processing is based on the AI model including a data-whitening model. Further, the pre-processing may include one or more of a data normalization, a noise-removal, and an equalization. Further, the 2400 may include a step 2408 of generating, using the processing device 704, the brain-wave dataset based on the pre-processing of the two or more datasets. Further, the brain-wave dataset comprises a combine dataset.

In some embodiments, the two or more datasets may include one or more of a clinical the clinical data, the demographic data, the patient-specific electronic medical record (EMR), and the patient-specific electric health record (EHR).

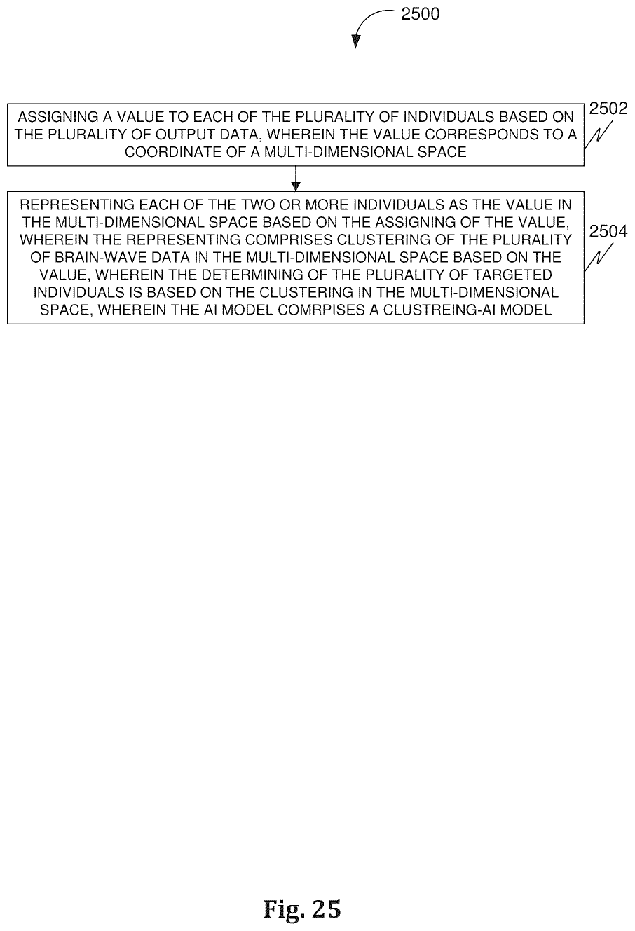

FIG. 25 illustrates a flowchart of a method 2500 of EEG-based biomarker discovery and commercialization for personalized diagnosis and treatment of brain disorders including clustering the plurality of brain-wave data in a multi-dimensional space, in accordance with some embodiments.

Further, in some embodiments, the method 2500 may include a step 2502 of assigning a value to each of the two or more individuals based on the two or more output data. Further, the value corresponds to a coordinate of a multi-dimensional space. Further, the method 2500 may include a step 2504 of representing each of the two or more individuals as the value in the multi-dimensional space based on the assigning of the value. Further, the representing comprises clustering of two or more brain-wave data in the multi-dimensional space based on the value. Further, the determining of the two or more targeted individuals may be based on the clustering in the multi-dimensional space. Further, the AI model comprises a clustering-AI model.

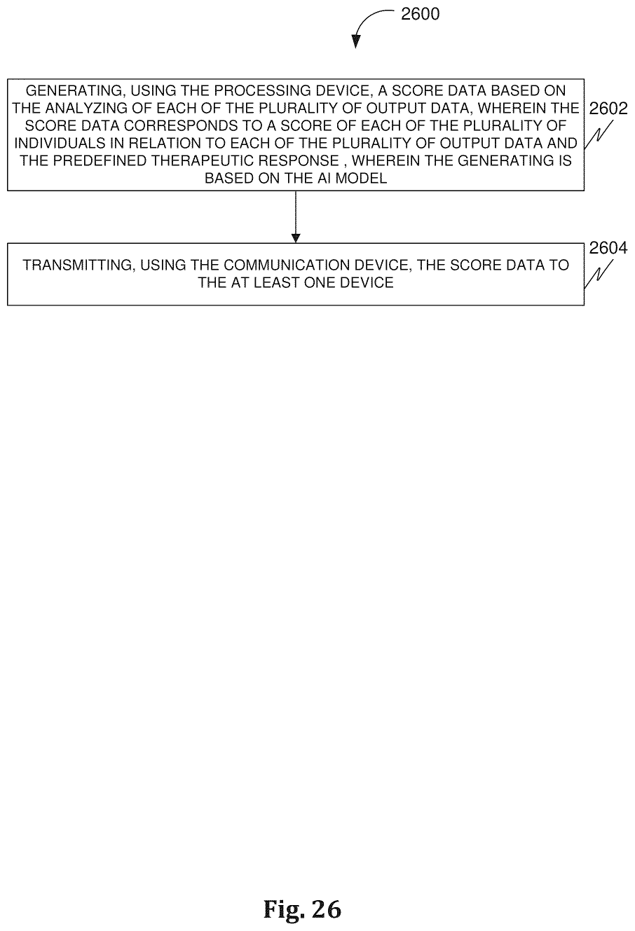

FIG. 26 illustrates a flowchart of a method 2600 of EEG-based biomarker discovery and commercialization for personalized diagnosis and treatment of brain disorders including generating, using the processing device 704, a score data, in accordance with some embodiments.

Further, in some embodiments, the method 2600 may include a step 2602 of generating, using the processing device 704, a score data based on the analyzing of each of the two or more output data. Further, the score data corresponds to a score of each of the two or more individuals in relation to each of the two or more output data and the predefined therapeutic response. Further, the generating is based on the AI model. Further, the method 2600 may include a step 2604 of transmitting, using the communication device 702, the score data to the at least one device.

In some embodiments, the AI model comprises a placebo non-responder AI model. Further, the score comprises a placebo score.

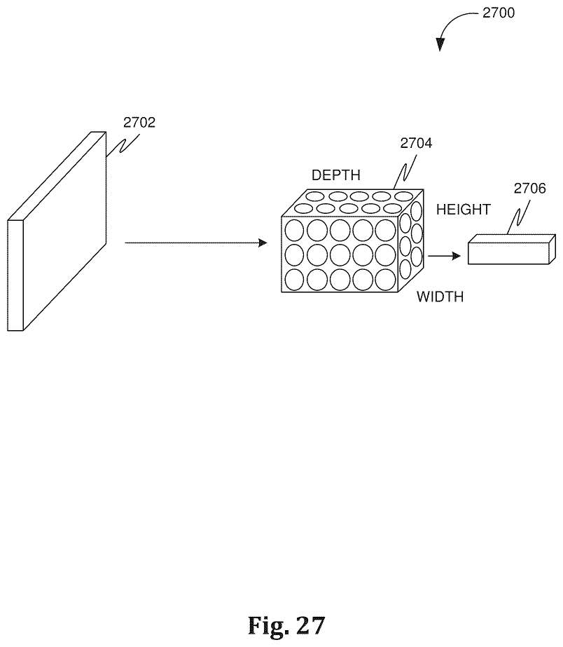

FIG. 27 illustrates a convolutional neural network 2700, according to an exemplary embodiment.

In some embodiments, the system and method may utilize a convolutional neural network or a deep learning neural network as the basis of the machine learning models.

In some embodiments, the convolutional neural network 2700 is particularly well suited to classifying features in data sets modelled in two or three dimensions. This makes CNNs popular for image classification, because images can be represented in computer memories in three dimensions (two dimensions for width and height, and a third dimension for pixel features like color components and intensity). For example, a color JEG image of size 480×480 pixels can be modelled in computer memory using an array that is 480×480×3, where each of the values of the third dimension is a red, green, or blue color component intensity for the pixel ranging from 0 to 255. Inputting this array of numbers to a trained CNN will generate outputs that describe the probability of the image being a certain class (0.80 for cat, 0.15 for dog, 0.05 for bird, etc.). Image classification is the task of taking an input image and outputting a class (a cat, dog, etc.) or a probability of classes that best describe the image.

In some embodiments, CNNs input the data set, pass it through a series of convolutional transformations, nonlinear activation functions (e.g., RELU), and pooling operations (downsampling, e.g., maxpool), and an output layer (e.g., softmax) to generate the classifications.

In some embodiments, the convolutional neural network 2700 arranges its neurons in three dimensions (width, height, depth), as visualized in a convolutional layer 2702. Every layer of the convolutional neural network 2700 transforms a 3D volume of inputs to a 3D output volume of neuron activations. In this example, an input layer 2704 encodes the image, so its width and height would be dimensions of an image, and the depth would be 3 (Red, Green, Blue channels). The convolutional layer 2702 further transforms outputs of the input layer 2704, and an output layer 2706 transforms the outputs of the convolutional layer 2702 into one or more classifications of the image content.

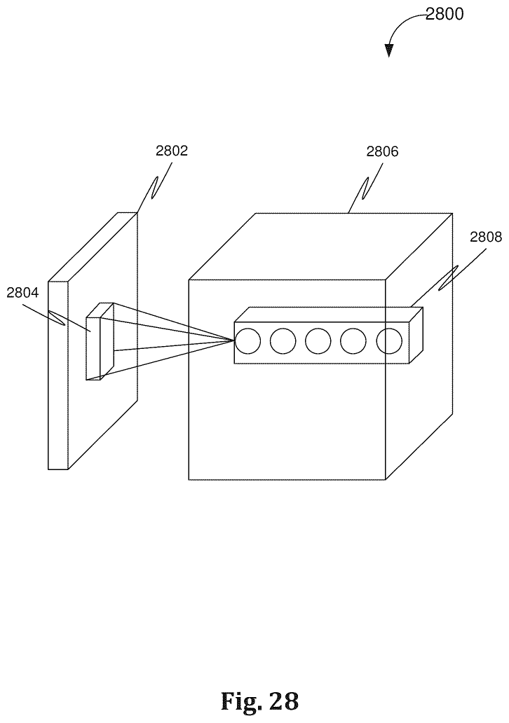

FIG. 28 illustrates a convolutional neural network layers 2800, in accordance with an exemplary embodiment. An example subregion of an input layer region 2804 of an input layer region 2802 of an image is analyzed by a set of convolutional layer subregion 2808 in the convolutional layer 2806. The input layer region 2802 is 32×32 neurons long and wide (e.g., 32×32 pixels), and three neurons deep (e.g., three color channels per pixel). Each neuron in the convolutional layer 2806 is connected only to a local region in the input layer region 2802 spatially (in height and width), but to the full depth (i.e., all color channels if the input is an image). Note, there are multiple neurons (5 in this example) along the depth of the convolutional layer subregion 2808 that analyzes the subregion of the input layer region 2804 of the input layer region 2802, in which each neuron of the convolutional layer subregion 2808 may receive inputs from every neuron of the subregion of the input layer region 2804.

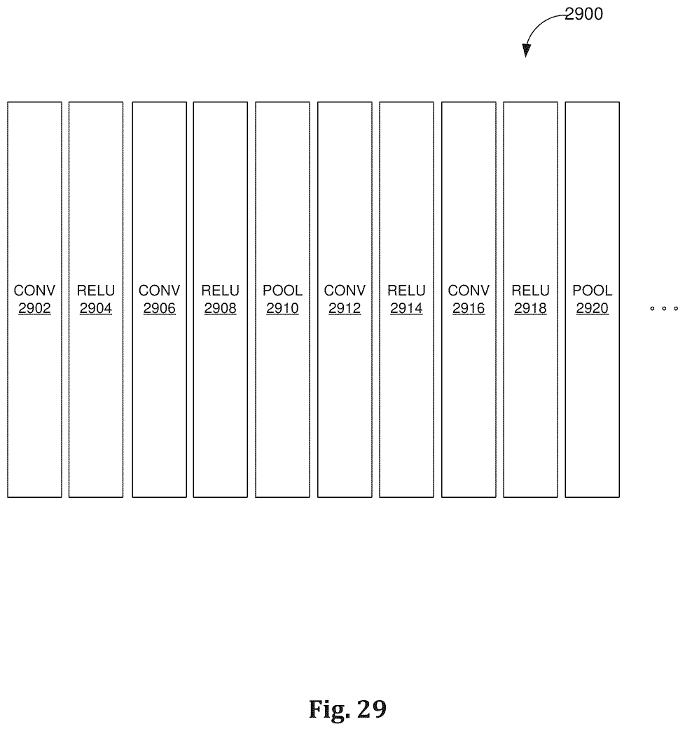

FIG. 29 illustrates a VGG net 2900, in accordance with some embodiments. Further, the initial convolution layer 2902 stores the raw image pixels, and the final pooling layer 2920 determines class scores. Each of the intermediate convolution layers (convolution layer 2906, convolution layer 2908, and convolution layer 2914) and rectifier activations (RELU layer 2904, RELUlayer 2910, RELUlayer 2912, and RELUlayer 2918) and intermediate pooling layers (pooling layer 2916, pooling layer 2920) along the processing path is shown as a column. Further, the VGG net 2900 replaces the large single-layer filters of basic CNNs with multiple 3×3 sized filters in series. With a given receptive field (the effective area size of input image on which output depends), multiple stacked smaller size filters may perform better at image feature classification than a single layer with a larger filter size, because multiple non-linear layers increase the depth of the network which enables it to learn more complex features. In a VGG net 2900 each pooling layer may be only 2×2.

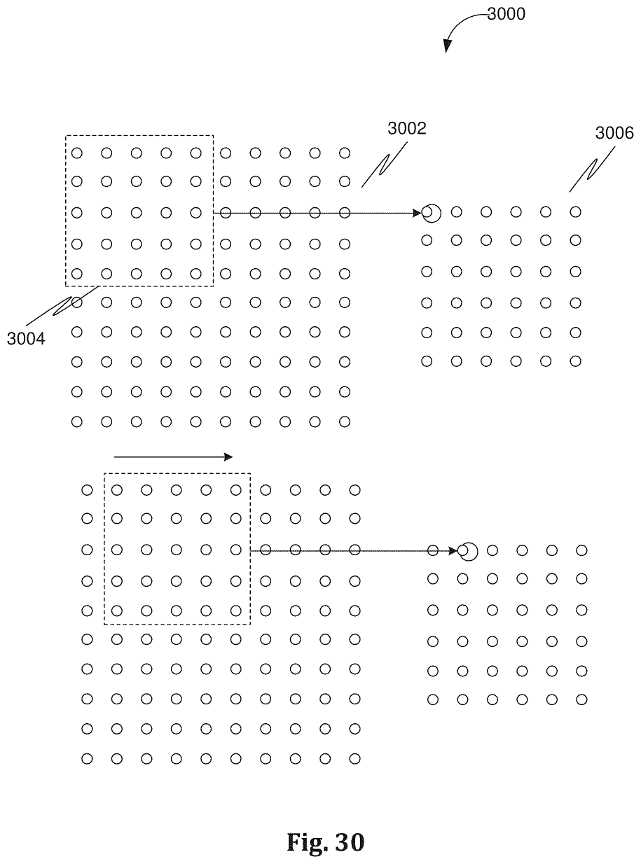

FIG. 30 illustrates a convolution layer filtering 3000 that connects outputs from groups of neurons in a convolution layer 3002 to neurons in a next layer 3006. A receptive field is defined for the convolution layer 3002, in this example, sets of 5×5 neurons. The collective outputs of each neuron in the receptive field are weighted and mapped to a single neuron in the next layer 3006. This weighted mapping is referred to as the filter 3004 for the convolution layer 3002 (or sometimes referred to as the kernel of the convolution layer 3002). The filter 3004 depth is not illustrated in this example (i.e., the filter 3004 is actually a cubic volume of neurons in the convolution layer 3002, not a square as illustrated). Thus, what is shown is a “slice” of the full filter 3004. The filter 3004 is slid, or convolved, around the input image, each time mapping to a different neuron in the next layer 3006. For example, FIG. 30 shows how the filter 3004 is stepped to the right by 1 unit (the “stride”), creating a slightly offset receptive field from the top one, and mapping its output to the next neuron in the next layer 3006. The stride can be and often is other numbers besides one, with larger strides reducing the overlaps in the receptive fields, and hence further reducing the size of the next layer 3006. Every unique receptive field in the convolution layer 3002 that can be defined in this stepwise manner maps to a different neuron in the next layer 3006. Thus, if the convolution layer 3002 is 32×32×3 neurons per slice, the next layer 3006 needs only to be 28×28×1 neurons to cover all the receptive fields of the convolution layer 3002. This is referred to as an activation map or feature map. There is thus a reduction in layer complexity from the filtering. There are 784 different ways that a 5×5 filter can uniquely fit on a 32×32 convolution layer 3002, so the next layer 1606 needs only be 28×28. The depth of the convolution layer 3002 is also reduced from 3 to 1 in the next layer 3006.

In some embodiments, number of total layers to use in a CNN, number of convolution layers, filter sizes, and values for strides at each layer are examples of “hyperparameters” of the CNN.

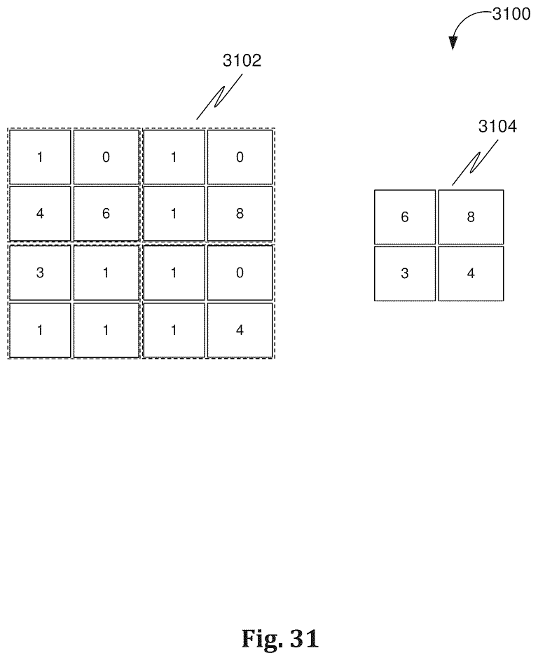

FIG. 31 illustrates a pooling layer function 3100 with a 2×2 receptive field and a stride of two. The pooling layer function 3100 is an example of maxpool pooling technique. Outputs of all the neurons in a particular receptive field of the input layer 3102 are replaced by the maximum valued one of those outputs in the pooling layer 3104. Other options for pooling layers are average pooling and L2-norm pooling. The reason to use a pooling layer is that once a specific feature is recognized in the original input volume (there will be a high activation value), its exact location is not as important as its relative location to the other features. Pooling layers can drastically reduce the spatial dimension of the input layer 3102 from that point forward in the neural network (the length and the width change, but not the depth). This serves two main purposes. The first is that the number of parameters or weights is greatly reduced thus lessening the computation cost. The second is that it will control overfitting. Overfitting refers to when a model is so tuned to the training examples that it is not able to generalize well when applied to live data sets.

Although the invention has been explained in relation to its preferred embodiment, it is to be understood that many other possible modifications and variations can be made without departing from the spirit and scope of the invention as hereinafter claimed.

What is claimed is:

1. A method of EEG-based biomarker discovery and commercialization for personalized diagnosis and treatment of brain disorders, the method comprising:
receiving, using a communication device, a brain-wave data from a diagnostic device, wherein the brain-wave data corresponds to a graphical representation of an electrical activity of a brain of an individual, wherein the individual is receiving at least one therapeutic for at least one brain disorder;
analyzing, using a processing device, the brain-wave data based on an artificial intelligence (AI) model, wherein the AI model is trained on a brain-wave dataset associated with a mental disorder, a psychiatric disorder, a neurological disorder, and a neurodegenerative disorder, and at least one additional data comprising a clinical data, a demographic data, an EMR (electronic medical record)/EHR (electronic health record) data, of the individual;
generating, using the processing device, at least one output data based on the analyzing, wherein the at least one output data is indicative of a response of the individual to the at least one therapeutic, and an influence of the at least one therapeutic on the individual; and
transmitting, using the communication device, the at least one data to at least one device.

2. The method of claim 1 further comprising:
pre-processing, using the processing device, the brain-wave data using at least one algorithm to obtain a pre-processed brain-wave data, wherein the pre-processing removes a noise-wave data from the brain-wave data, wherein the brain-wave data comprises the noise-wave data, wherein the pre-processing is based on the AI model; and
analyzing, using the processing device, the pre-processed brain-wave data based on the AI model, wherein the generating of the at least one output data is further based on the analyzing of the pre-processed brain-wave data.

3. The method of claim 1, wherein the brain-wave data comprises a plurality of brain-wave data corresponding to a plurality of individuals, wherein the at least one output data comprises a plurality of output data corresponding to the plurality of individuals, wherein the plurality of individuals is associated with a clinical trial of the at least one therapeutic, wherein the method further comprises:
analyzing, using the processing device, each of the plurality of output data based on a predefined therapeutic response, wherein the predefined therapeutic response influences an outcome of the clinical trial, wherein the analyzing of each of the plurality of output data is based on the AI model;
determining, using the processing device, a plurality of targeted individuals from the plurality of individuals based on the analyzing of each of the plurality of output data;
generating, using the processing device, a report data based on the determining of the plurality of targeted individuals, wherein the report data corresponds to a report associated with the plurality of target individuals; and
transmitting, using the communication device, the report data to the at least one device.

4. The method of claim 3, wherein the analyzing of each of the plurality of output data comprises:
assigning a value to each of the plurality of individuals based on the plurality of output data, wherein the value corresponds to a coordinate of a multi-dimensional space; and
representing each of the plurality of individuals as the value in the multi-dimensional space based on the assigning of the value, wherein the determining of the plurality of targeted individuals is based on a proximity between a plurality of values associated with the plurality of individuals in the multi-dimensional space.

5. The method of claim 3, wherein the plurality of targeted individuals comprises at least one of a placebo responder and a non-placebo responder, wherein the predefined therapeutic response comprises at least one of a placebo response and a non-placebo response, wherein the placebo responder experiences a placebo-recovery from the at least one brain disorder, wherein the placebo-recovery is associated with a placebo effect, wherein the non-placebo responder experiences a therapeutic-recovery from the at least one brain disorder, wherein the therapeutic-recovery is associated with an active treatment of the at least one therapeutic, wherein the report comprises at least one of a placebo responder report and a non-placebo responder report.

6. The method of claim 1, wherein the analyzing of the brain-wave data comprises identifying a biological-characteristic of the individual, wherein the electrical activity of the brain is based on the biological-characteristic, wherein the at least one brain disorder is associated with the biological-characteristic, wherein the biomarker is associated with the biological-characteristic.

7. The method of claim 1, wherein the brain-wave dataset comprises each of a target-labelled brain-wave data and a target-unlabeled brain-wave data, wherein each of the target-labelled brain-wave data and the target-unlabeled brain-wave data is recorded from a plurality of drug-trail participants, wherein the plurality of drug-trail participants is associated with a clinical trial of the at least one therapeutic comprising at least one of a placebo treatment and an active treatment, wherein the target-labelled brain-wave data comprises an indicator indicating the at least one therapeutic associated with each of the plurality of drug-trail participants, wherein the target-unlabeled brain-wave data lacks the indicator.

8. The method of claim 1, wherein the AI model comprises a plurality of AI models comprising each of the first AI model, a second AI model, a third AI model, and a fourth AI model, wherein the first AI model is configured to be trained on a target-unlabeled brain-wave data based on a self-supervised learning technique, wherein the brain-wave dataset comprises the target-unlabeled brain-wave data, wherein the second AI model is configured for clustering a plurality of individuals in a multi-dimensional space, wherein the plurality of individuals is associated with the target-unlabeled brain-wave data.

9. The method of claim 8, wherein the second AI model is further configured for predicting a biological characteristic associated with each of the plurality of individuals, wherein the third AI model is configured for predicting a response of the plurality of individuals to a therapy, wherein the fourth AI model is configured for predicting a therapeutic response of the plurality of individuals to the at least one therapeutic, wherein the fourth AI model is configured to be trained using a supervised learning.

10. The method of claim 9, wherein the AI model is based on at least one of a deep convolutional neural network and a generative adversarial network.

11. A system of EEG-based biomarker discovery and commercialization for personalized diagnosis and treatment of brain disorders, the system comprising:
a communication device configured for:
receiving a brain-wave data from a diagnostic device, wherein the brain-wave data corresponds to a graphical representation of an electrical activity of a brain of an individual, wherein the individual is receiving at least one therapeutic for at least one brain disorder;
transmitting at least one output data to at least one device; and
a processing device communicatively coupled with the communication device, wherein the processing device is configured for:
analyzing the brain-wave data based on an artificial intelligence (AI) model, wherein the AI model is trained on a brain-wave dataset associated with a mental disorder, a psychiatric disorder, a neurological disorder, and a neurodegenerative disorder, and at least one additional data comprising a clinical data, a demographic data, an EMR (electronic medical record)/EHR (electronic health record) data, of the individual; and
generating the at least one output data based on the analyzing, wherein the at least one output data is indicative of a response of the individual to the at least one therapeutic, and an influence of the at least one therapeutic on the individual.

12. The system of claim 11, wherein the processing device is further configured for:
pre-processing the brain-wave data using at least one algorithm to obtain a pre-processed brain-wave data, wherein the pre-processing removes a noise-wave data from the brain-wave data, wherein the brain-wave data comprises the noise-wave data, wherein the pre-processing is based on the AI model; and
analyzing the pre-processed brain-wave data based on the AI model, wherein the generating of the at least one output data is further based on the analyzing of the pre-processed brain-wave data.

13. The system of claim 11, wherein the brain-wave data comprises a plurality of brain-wave data corresponding to a plurality of individuals, wherein the at least one output data comprises a plurality of output data corresponding to the plurality of individuals, wherein the plurality of individuals is associated with a clinical trial of the at least one therapeutic, wherein the processing device is further configured for:
analyzing each of the plurality of output data based on a predefined therapeutic response, wherein the predefined therapeutic response influences an outcome of the clinical trial, wherein the analyzing of each of the plurality of output data is based on the AI model;
determining a plurality of targeted individuals from the plurality of individuals based on the analyzing of each of the plurality of output data; and
generating a report data based on the determining of the plurality of targeted individuals, wherein the report data corresponds to a report associated with the plurality of target individuals, wherein the communication device is further configured for transmitting the report data to the at least one device.

14. The system of claim 13, wherein the analyzing of each of the plurality of output data comprises:
assigning a value to each of the plurality of individuals based on the plurality of output data, wherein the value corresponds to a coordinate of a multi-dimensional space; and
representing each of the plurality of individuals as the value in the multi-dimensional space based on the assigning of the value, wherein the determining of the plurality of targeted individuals is based on a proximity between a plurality of values associated with the plurality of individuals in the multi-dimensional space.

15. The system of claim 13, wherein the plurality of targeted individuals comprises at least one of a placebo responder and a non-placebo responder, wherein the predefined therapeutic response comprises at least one of a placebo response and a non-placebo response, wherein the placebo responder experiences a placebo-recovery from the at least one brain disorder, wherein the placebo-recovery is associated with a placebo effect, wherein the non-placebo responder experiences a therapeutic-recovery from the at least one brain disorder, wherein the therapeutic-recovery is associated with an active treatment of the at least one therapeutic, wherein the report comprises at least one of a placebo responder report and a non-placebo responder report.

16. The system of claim 11, wherein the analyzing of the brain-wave data comprises identifying a biological-characteristic of the individual, wherein the electrical activity of the brain is based on the biological-characteristic, wherein the at least one brain disorder is associated with the biological-characteristic, wherein the biomarker is associated with the biological-characteristic.

17. The system of claim 11, wherein the brain-wave dataset comprises each of a target-labelled brain-wave data and a target-unlabeled brain-wave data, wherein each of the target-labelled brain-wave data and the target-unlabeled brain-wave data is recorded from a plurality of drug-trail participants, wherein the plurality of drug-trail participants is associated with a clinical trial of the at least one therapeutic comprising at least one of a placebo treatment and an active treatment, wherein the target-labelled brain-wave data comprises an indicator indicating the at least one therapeutic associated with each of the plurality of drug-trail participants, wherein the target-unlabeled brain-wave data lacks the indicator.

18. The system of claim 11, wherein the AI model comprises a plurality of AI models comprising each of the first AI model, a second AI model, a third AI model, and a fourth AI model, wherein the first AI model is configured to be trained on a target-unlabeled brain-wave data based on a self-supervised learning technique, wherein the brain-wave dataset comprises the target-unlabeled brain-wave data, wherein the second AI model is configured for clustering a plurality of individuals in a multi-dimensional space, wherein the plurality of individuals is associated with the target-unlabeled brain-wave data.

19. The system of claim 18, wherein the second AI model is further configured for predicting a biological characteristic associated with each of the plurality of individuals, wherein the third AI model is configured for predicting a response of the plurality of individuals to a therapy, wherein the fourth AI model is configured for predicting the therapeutic response of the plurality of individuals to the at least one therapeutic, wherein the fourth AI model is configured to be trained using a supervised learning.

20. The system of claim 19, wherein the AI model is based on at least one of a deep convolutional neural network and a generative adversarial network.
